SEQUENCE LISTING (7 sequences)

SEQ ID NO: 1 - DNA, 567 bp, Artificial Sequence
  Optimized human Interferon alpha 2b
atggccctgaccttcgccctgctggtggccctgctggtgctgtcctgcaagagcagctgcagcgtgggctgcgacctgccccagacccacagcctgggcagccggcggaccctgatgctgctggcccagatgcggcggatcagcctgttcagctgcctgaaggaccggcacgacttcggcttcccccaggaagagttcggcaaccagttccagaaggccgagaccatccccgtgctgcacgagatgatccagcagatcttcaacctgttcagcaccaaggacagcagcgccgcctgggacgagaccctgctggacaagttctacaccgagctgtaccagcagctgaacgacctggaagcctgcgtgatccagggcgtgggcgtgaccgagacccccctgatgaaagaggacagcatcctggccgtgcggaagtacttccagcggatcaccctgtacctgaaagagaagaagtacagcccctgcgcctgggaagtggtgcgggccgagatcatgcggagcttcagcctgagcaccaacctgcaggaaagcctgcggagcaaagagtga

SEQ ID NO: 2 - DNA, 5246 bp, Artificial Sequence
  Complete sequence encoding Optimized human Interferon alpha 2b with CAG Promoter in pAdeno shuttle
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggaggcgcgccctcgagctagcccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgattaattcgagcgaacgcgtcgagtcgctcggtacgatttaaattgaattgggctcgagatctgcgatctaagtaagcttgcatgcctgcaggtcgactctagactgccatggccctgaccttcgccctgctggtggccctgctggtgctgtcctgcaagagcagctgcagcgtgggctgcgacctgccccagacccacagcctgggcagccggcggaccctgatgctgctggcccagatgcggcggatcagcctgttcagctgcctgaaggaccggcacgacttcggcttcccccaggaagagttcggcaaccagttccagaaggccgagaccatccccgtgctgcacgagatgatccagcagatcttcaacctgttcagcaccaaggacagcagcgccgcctgggacgagaccctgctggacaagttctacaccgagctgtaccagcagctgaacgacctggaagcctgcgtgatccagggcgtgggcgtgaccgagacccccctgatgaaagaggacagcatcctggccgtgcggaagtacttccagcggatcaccctgtacctgaaagagaagaagtacagcccctgcgcctgggaagtggtgcgggccgagatcatgcggagcttcagcctgagcaccaacctgcaggaaagcctgcggagcaaagagtgaggatccccgggtaccgagctcgaattctttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcgatatcggcgcgccgggcccctacgtcacccgccccgttcccacgccccgcgccacgtcacaaactccaccccctcattatcatattggcttcaatccaaaataaggtatattattgatgatggccgcagcggcccctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggggccgctgcggc

SEQ ID NO: 3 - DNA, 4669 bp, Artificial Sequence
  Sequence form Adenovirus, CMV and variation of human IFN alpha
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggaggcgcgccctcgagctagctgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtaagcttgcatgcctgcaggtcgactctagactgccatggccctgaccttcgccctgctggtggccctgctggtgctgtcctgcaagagcagctgcagcgtgggctgcgacctgccccagacccacagcctgggcagccggcggaccctgatgctgctggcccagatgcggcggatcagcctgttcagctgcctgaaggaccggcacgacttcggcttcccccaggaagagttcggcaaccagttccagaaggccgagaccatccccgtgctgcacgagatgatccagcagatcttcaacctgttcagcaccaaggacagcagcgccgcctgggacgagaccctgctggacaagttctacaccgagctgtaccagcagctgaacgacctggaagcctgcgtgatccagggcgtgggcgtgaccgagacccccctgatgaaagaggacagcatcctggccgtgcggaagtacttccagcggatcaccctgtacctgaaagagaagaagtacagcccctgcgcctgggaagtggtgcgggccgagatcatgcggagcttcagcctgagcaccaacctgcaggaaagcctgcggagcaaagagtgaggatccccgggtaccgagctcgaattctttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcgatatcggcgcgccgggcccctacgtcacccgccccgttcccacgccccgcgccacgtcacaaactccaccccctcattatcatattggcttcaatccaaaataaggtatattattgatgatggccgcagcggcccctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggggccgctgcggc

SEQ ID NO: 4 - DNA, 645 bp, Homo sapiens
ggcgcgccaagcttgcatgcctgcaggtcgactctagactgccatggccttgacctttgctttactggtggccctcctggtgctcagctgcaagtcaagctgctctgtgggctgtgatctgcctcaaacccacagcctgggtagcaggaggaccttgatgctcctggcacagatgaggagaatctctcttttctcctgcttgaaggacagacatgactttggatttccccaggaggagtttggcaaccagttccaaaaggctgaaaccatccctgtcctccatgagatgatccagcagatcttcaatctcttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattctggctgtgaggaaatacttccaaagaatcactctctatctgaaagagaagaaatacagcccttgtgcctgggaggttgtcagagcagaaatcatgagatctttttctttgtcaacaaacttgcaagaaagtttaagaagtaaggaatgaggatccccgggtaccgagctcgaattcttaattaa

SEQ ID NO: 5 - protein, 188 aa, Homo sapiens
MALTFALLVALLVLSCKSSCSVGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 6 - DNA, 1180 bp, Artificial Sequence
  CAG promoter
ccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgattaattcgagcgaacgcgtcgagtcgctcggtacgatttaaattgaattgggctcgagatctgcgatctaagt

SEQ ID NO: 7 - DNA, 203 bp, Artificial Sequence
  SV40 poly A sequence
tttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatc

(19) United States
(12) Patent Application Publication
(10) Pub. No.: US 2013/0171107 A1
(43) Pub. Date: Jul. 4, 2013

(54) LONG LASTING DRUG FORMULATIONS

(71) Applicant: Medgenics Medical Israel Ltd., Misgav (IL)

(72) Inventors: Andrew L. Pearlman, D.N. Misgav (IL); Baruch S. Stern, Haifa (IL); Nir Shapir, Misgav (IL)

(73) Assignee: MEDGENICS MEDICAL ISRAEL LTD., Misgav (IL)

(21) Appl. No.: 13/794,845

(22) Filed: Mar. 12, 2013

Related U.S. Application Data
(63) Continuation-in-part of application No. 13/172,915, filed on Jun. 30, 2011.
Application No. 13/172,915 is a division of application No. 11/898,481, filed on Sep. 12, 2007.
(60) Provisional application No. 60/844,351, filed on Sep. 14, 2006.

(51) Int. Cl. A61K 48/00

(52) U.S. Cl. CPC A61K 48/00
USPC 424/93.2

(57) ABSTRACT

The present invention is directed to long-lasting therapeutic formulations and their methods of use wherein the formulation comprises a genetically modified micro-organ that comprises a vector which comprises a nucleic acid sequence operably linked to one or more regulatory sequences, wherein the nucleic acid sequence encodes a therapeutic polypeptide, such as erythropoietin or interferon alpha.

### CROSS REFERENCE TO RELATED APPLICATIONS

This application is a continuation in part application of U.S. application Ser. No. 13/172,915, filed Jun. 30, 2011, which is a divisional application of U.S. application Ser. No. 11/898,481, filed Sep. 12, 2007, which claims the benefit of U.S. Patent Application Ser. No. 60/844,351, filed on Sep. 14, 2006; and claims the benefit of U.S. Patent Application Ser. Nos. 61/635,614, filed Apr. 19, 2012 and 61/695,115, filed Aug. 30, 2012, which are all incorporated in their entirety herein by reference.

### FIELD OF INVENTION

This invention is directed to long-lasting therapeutic formulations comprising a genetically modified, tissue-based micro-organ comprising a vector comprising a nucleic acid sequence encoding a therapeutic polypeptide, such as erythropoietin or interferon alpha, operably linked to one or more regulatory sequences and their methods of use.

### BACKGROUND OF THE INVENTION

Therapeutic agents can be delivered orally, transdermally, by inhalation, by injection or by depot with slow release. However, the method of delivery is limited by the processing that the agent is subjected to in the recipient, by the requirement for frequent administration, and limitations on the size of molecules that can be utilized. For some of the methods, the amount of therapeutic agent varies between administrations.

Protein production techniques which involve the sub-cloning of a desired nucleic acid sequence/fragment into a vector which is subsequently used for modifying specific host cells, which are meant to produce the desired protein for further purification steps are limited in the amount of protein expressed, protein secretion, post-translational modifications (such as glycosylation and the accurate folding of the protein), etc. Moreover, even if a high-level of protein production could be achieved, large quantities of the recombinant protein must then be produced and purified to be free of contaminants Development of a purification scheme is a very lengthy process. And once purified recombinant protein has been obtained, it must be further formulated to render it stable and acceptable for introduction into animals or humans. Furthermore, even formulated, purified recombinant proteins have a finite shelf life due to maintenance and storage limitations; often requiring repeated purification and formulation of more protein. The process of developing an appropriate formulation is time consuming, difficult, and costly, as well.

Thus, there is a widely recognized need for long-lasting protein-based therapeutic molecules that have the requisite post-translational modifications to preserve their biological activity, which are produced inexpensively and quickly without the need for the laborious and costly methods typically associated with obtaining high-levels of recombinant proteins.

Some researchers have attempted to obtain in vivo expression of recombinant gene products via gene therapy. Typically viral vectors are used to transduce cells in vivo to express recombinant gene products. These viral-based vectors have advantageous characteristics, such as the natural ability to infect the target tissue. However, retrovirus-based vectors require integration within the genome of the target tissue to allow for recombinant product expression (with the potential to activate resident oncogenes) and can only be used to transduce actively dividing tissues. Viral vectors are also often no able to sustain long-term transgene expression, which may be due at least in part to their elimination due to secondary host immune responses.

Accordingly, there remains a need in the art for recombinant gene product formulations that have consistently high expression levels lasting for several weeks or more and for methods of using those formulations to treat disease.

### SUMMARY OF THE INVENTION

The invention provides, in one embodiment, a long-lasting therapeutic interferon formulation comprising at least one genetically modified micro-organ that expresses and secretes interferon, said genetically modified micro-organ comprising a nucleic acid sequence encoding an interferon operably linked to one or more regulatory sequences, wherein said nucleic acid encoding interferon comprises SEQ ID No. 1. In one embodiment, the at least one genetically modified micro-organ is a genetically modified dermal micro-organ. In one embodiment, the helper-dependent adenoviral vector comprises SEQ ID No. 2.

This invention provides in some embodiments, a method of treating hepatitis in a human subject in need over a sustained period of time comprising the steps of: (i) providing at least one genetically modified micro-organ that expresses and secretes interferon, said genetically modified micro-organ comprising a nucleic acid sequence encoding interferon operably linked to one or more regulatory sequences; (ii) determining interferon secretion levels of said at least on genetically modified micro-organ in vitro; (iii) implanting said at least one genetically modified micro-organ in said human subject at an effective dosage; and either (iv) measuring interferon in the serum of said human subject, or (v) measuring levels of hepatic virus in said human subject. In one embodiment, a method of this invention treats a subject suffering from hepatitis, for example, hepatitis B, hepatitis C or hepatitis D, or any combination thereof. In one embodiment, a method of this invention treats a subject suffering from hepatitis C who is infected with genotype 1 virus. In another embodiment, the subject in need suffering from hepatitis C is infected with genotype 2 virus. In yet another embodiment, the subject in need suffering from hepatitis C is infected with genotype 3 virus. In still another embodiment, the subject in need suffering from hepatitis C is infected with a combination of virus genotypes.

In some embodiments, methods of this invention treat hepatitis in a subject, wherein said hepatitis is hepatitis D. In one embodiment, the hepatitis D is chronic hepatitis D.

In one embodiment, a genetically modified micro-organ used in the methods of this invention comprise a nucleic acid sequence encoding interferon that is optimized for increased expression levels, increased duration of expression, or a combination thereof. In one embodiment, the optimized nucleic acid sequence is greater than 85% homologous to SEQ ID No. 1. In one embodiment, the helper dependent adenovirus vector comprises SEQ ID No. 2.

In some embodiments of this invention, the genetically modified micro-organ used in the methods of this invention is a genetically modified dermal micro-organ.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 presents levels of recombinant optimized human interferon-alpha (IFNα) produced in vitro by the formulations of the instant invention comprising a CAG promoter;

FIG. 2 presents levels of recombinant human erythropoietin (hEPO) produced in vitro by the formulations of the instant invention. HD-Ad-CAG-wt-hEPO GMMO titration (A). Micro-organs were transduced with increasing dilutions of HD-Ad-CAG-wt-hEPO virus: 1:25; 1:100; and 1:1000 dilutions. Ad5/CMV/wt-hEPO was diluted to a working concentration of 1:10 and 1:50. A comparison between GMMOs produced from two different skins, H-1 and H-2 (B). Micro-organs were transduced with HD-Ad-CAG-wt-hEPO 1:25. Bars indicate the hEPO concentration measured by ELISA in the culture media that was collected and replaced every 3-4 days;

FIG. 3 presents the percent of peak erythropoietin (EPO) expression levels in vitro from optimized formulations comprising EPO-expressing gutless adenovirus and micro-organs comprising EPO-expressing adenovirus-5. Micro-organs were transduced with HD-Ad-CAG-hEPO at 1:25 or with Ad5/CMV/hEPO at 1:10;

FIG. 4 presents erythropoietin (EPO) expression levels in vitro from formulations comprising optimized and non-optimized EPO-expressing gutless adenovirus. Micro-organs were transduced with a working dilution of 1:100 viral particles. Bars indicate the hEPO concentration measured by ELISA in the culture media that was collected and replaced every 3-4 days;

FIG. 5 presents erythropoietin (EPO) expression levels in vitro from formulations comprising EPO-expressing gutless adenovirus downstream of a CAG or CMV promoter;

FIG. 6 presents levels of recombinant human erythropoietin produced in vivo in SCID mice (A) and in vitro (B) by the formulations of the instant invention in vitro and the associated changes in hematocrit (A). Ten mice/group were implanted subcutaneously with GMMOs. The hEPO levels (mU/ml) and the corresponding % hematocrit that were measured in the serum of mice that were implanted with GMMOs transduced with adenovirus-hEPO, helper-dependent adenovirus-hEPO, and helper-dependent adenovirus-optimized hEPO and with non-transduced GMMOs are presented. Bleeds were done every 10 days (A). Hematocrit was measured by the centrifugation method and serum hEPO levels in the blood were measured by a hEPO ELISA kit. Non-implanted GMMOs were maintained in culture and levels of EPO were measured (B);

FIG. 7 presents sustained levels of IFNα production produced in vitro by an embodiment of a hIFN-Biopump of this invention;

FIG. 8 presents daily in vitro IFNα production on day 10 post harvesting from embodiments of Biopumps from seven individuals;

FIG. 9 presents potency adjustment by vector titration, wherein HDAd-IFNα titers corresponding to 3×10¹⁰ (leftmost bar within each grouping), 1.5×10¹⁰ (second to leftmost bar within each grouping), 5×10⁹ (second to rightmost bar within each grouping), and 1×10⁹ (rightmost bar within each grouping) vp/MO;

FIG. 10 presents micro-organ viability data by viable cell count per micro-organ following harvesting after 1 day compared with 9 days;

FIGS. 11A and 11B present IFN in vitro secretion levels from Biopumps produced from two different subjects (11A and 11B) transduced after 24 or 72 hours;

FIG. 12 presents dose dependent delivery of embodiments of hIFNα-Biopumps in SCID mice;

FIG. 13 presents secretion levels and corresponding activity levels of IFNα in SCID mice;

FIG. 14 presents correlation between serum IFNα by ELISA versus serum IFNα bioactivity in SCID mice;

FIG. 15 presents typical evolution of serological and virological markers in hepatitis D virus infection, based on Hughes S A, Wedemeyer H, Harrison P M. (2011) Hepatitis delta virus. Lancet 378(9785):73-85.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

In some embodiments, the instant invention is directed to long-lasting therapeutic formulations comprising a genetically modified, tissue-based micro-organ comprising a vector comprising a nucleic acid sequence encoding a therapeutic polypeptide, such as erythropoietin or interferon alpha, operably linked to one or more regulatory sequences and their methods of use.

The invention provides, in one embodiment, a long-lasting therapeutic formulation comprising a genetically modified micro-organ, said micro-organ comprising a vector comprising a nucleic acid sequence operably linked to one or more regulatory sequences, wherein said nucleic acid sequence encodes a therapeutic polypeptide and whereby the expression level of the therapeutic polypeptide is increased by more than 5% over basal level and said increase is maintained for greater than one month. In another embodiment, the expression level of the therapeutic polypeptide is increased by more than 5% over basal level and said increase is maintained for greater than six months.

In another embodiment, this invention provides a long-lasting therapeutic formulation comprising a genetically modified micro-organ, said micro-organ comprising a vector comprising a nucleic acid sequence operably linked to one or more regulatory sequences, wherein said nucleic acid sequence encodes a therapeutic polypeptide and whereby the expression level of the therapeutic polypeptide is increased by more than 5% over basal level and said increase is maintained for greater than one month and wherein said vector is a helper-dependent adenovirus vector.

In another embodiment, the invention provides a long-lasting therapeutic formulation comprising a genetically modified micro-organ, said micro-organ comprising a vector comprising a nucleic acid sequence operably linked to one or more regulatory sequences, wherein said nucleic acid sequence encodes a therapeutic polypeptide and whereby the expression level of the therapeutic polypeptide is increased by more than 5% over basal level and said increase is maintained for greater than one month in an immuno-competent host.

In another embodiment, the invention provides a long-lasting therapeutic formulation comprising a genetically modified micro-organ, said micro-organ comprising a vector comprising a nucleic acid sequence operably linked to one or more regulatory sequences, whereby the expression level of the therapeutic nucleic acid is increased by more than 5% over basal levels. In one embodiment, the expression level of the therapeutic nucleic acid is increased by more than 5% over basal levels in an immuno-competent host, while in another embodiment, said vector is a helper-dependent adenovirus vector.

In one embodiment, the invention provides a long-lasting therapeutic formulation and methods of use thereof where the formulation comprises a genetically modified micro-organ. In one embodiment, the term “micro-organ” as used herein, refers in one embodiment, to an isolated tissue or organ structure derived from or identical to an explant that has been prepared in a manner conducive to cell viability and function. In one embodiment, a micro-organ maintains at least some in vivo structures, or in another embodiment, interactions, similar to the tissues or organ from which it is obtained, In another embodiment, micro-organs retain the micro-architecture and the three dimensional structure of the tissue or organ from which they were derived and have dimensions selected so as to allow passive diffusion of adequate nutrients and gases to cells within the micro-organ and diffusion of cellular waste out of the cells of the micro-organ so as to minimize cellular toxicity and concomitant cell death due to insufficient nutrition and/or accumulation of waste. In one embodiment, a micro-organ is a sliver of dermal tissue.

In one embodiment, a micro-organ is 1-2 mm in diameter and 30-40 mm in length. In another embodiment, the diameter of a micro-organ may be, for example, 1-3 mm, 1-4 mm, 2-4 mm, 0.5-3.5 mm, or 1.5-10 mm. In another embodiment the diameter of a micro-organ may be, for example, approximately 2 mm or approximately 1.5 mm. In another embodiment, the length of the micro-organ may be 5-100 mm, 10-60 mm, 20-60 mm, 20-50 mm, 20-40 mm, 20-100 mm, 30-100 mm, 40-100 mm, 50-100 mm, 60-100 mm, 70-100 mm, 80-100 mm, or 90-100 mm. In another embodiment, the length of the micro-organ may be approximately 20 mm, approximately 30 mm, approximately 40 mm, or approximately 50 mm.

In another embodiment, a DMO can be distinguished from a collection of isolated cells, which in one embodiment, are grown on a natural or artificial scaffold, in that DMOs maintain the micro-architecture and the three dimensional structure of dermal tissue from which they were derived. Thus, in one embodiment, a DMO is not one or more cell types grown on a scaffold or within a gel. An advantage of a DMO of this invention over cells, a cell suspension, or cells incorporated in a natural or artificial scaffold wherein cells may migrate from the scaffold, is that a DMO can be implanted at a defined position in the body, so that if necessary it could be readily removed or ablated in the future. This allows for a reduction or cessation of treatment if necessary. This contrasts with cell suspension transplantation, transplantation of an artificial comprising tissue culture cells or primary culture cells, or implantation of an artificial scaffold comprising tissue culture cells or primary culture cells, into the body, in which the cells can migrate or become “lost” within tissues of the body. Further, cells incorporated in a natural or artificial scaffold lack the ability to provide paracrine signaling as cells are not necessary adjacent one to another. This contrasts the cells of a dermal micro-organ, which maintain the cell-cell interactions and three-dimensional structure and micro-architecture of dermal tissue. An additional advantage of a DMO is that it is primary tissue, which requires minimal ex-vivo processing to produce a genetically modified dermal micro-organ. Therefore, an autologous GM-DMO of this invention, for instance expressing IFN, would be recognized as “self” upon implantation, whereas an artificial scaffold would be recognized as a foreign body.

A detailed description of micro-organs can be found in US-2003-0152562, which is incorporated herein by reference in its entirety.

Earlier patents (WO 03/006669, WO 03/035851, WO 04/099363, which are incorporated herein by reference) described micro-organs, which can be modified to express a gene product of interest, that may be sustained outside the body in an autonomously functional state for an extended period of time, and may then be implanted subcutaneously or in other locations within the body for the purpose of treating diseases or disorders. However, the micro-organs of the present invention unexpectedly showed a much longer-term expression profile of a gene product of interest in vitro and in vivo.

As used herein, the term “explant” refers, in one embodiment, to a tissue or organ or a portion thereof removed from its natural growth site in an organism and placed in a culture medium for a period of time. In one embodiment, the tissue or organ is viable, in another embodiment, metabolically active, or a combination thereof.

As used herein, the term “microarchitecture” refers, in one embodiment, to a characteristic of the explant in which some or all of the cells of the tissue explant maintain, in vitro, physical and/or functional contact with at least one cell or non-cellular substance with which they were in physical and/or functional contact in vivo.

In another embodiment, micro-organ explants maintain the three-dimensional structure of the tissue or organ from which they were derived. In one embodiment, micro-organ explants retain the spatial interactions, e.g. cell-cell, cell-matrix and cell-stromal interactions, and the orientation of the tissue from which they were derived. In one embodiment, preservation of spatial interactions such as described above permit the maintenance of biological functions of the explant, such as secretion of autocrine and paracrine factors and other extracellular stimuli, which in one embodiment, provide long term viability to the explant. In one embodiment, at least some of the cells of the micro-organ explant maintain, in vitro, their physical and/or functional contact with at least one cell or non-cellular substance with which they were in physical and/or functional contact in vivo. In one embodiment, some of the cells refers to at least about 50%, in another embodiment, at least about 60%, in another embodiment at least about 70%, in another embodiment, at least about 80%, and in another embodiment, at least about 90% or more of the cells of the population. In another embodiment, the cells of the explant maintain at least one biological activity of the organ or tissue from which they are isolated.

In one embodiment, the term “about”, refers to a deviance of between 0.0001-5% from the indicated number or range of numbers. In one embodiment, the term “about”, refers to a deviance of between 1-10% from the indicated number or range of numbers. In one embodiment, the term “about”, refers to a deviance of up to 25% from the indicated number or range of numbers.

In some embodiments, any of the formulation of this invention will comprise a genetically modified micro-organ, in any form or embodiment as described herein. As used herein, the terms “genetically modified micro-organ”, “genetically modified dermal micro-organ” and “Biopump” are interchangeable and refer in one embodiment to a micro-organ, which may be a dermal micro-organ, genetically modified to express a recombinant gene product.

In one embodiment, this invention provides a “genetically modified dermal micro-organ” (“GM-DMO”). In one embodiment, a DMO is modified to express at least a gene product of interest, wherein the DMO may then be considered a therapeutic dermal micro-organ. In one embodiment, a therapeutic DMO of this invention expresses and secretes an IFN polypeptide or functional fragment thereof.

In some embodiments, any of the formulations of this invention will consist of a genetically modified micro-organ, in any form or embodiment as described herein. In some embodiments, of the compositions of this invention will consist essentially of a genetically modified micro-organ, in any form or embodiment as described herein. In some embodiments, the term “comprise” refers to the inclusion of the indicated active agent, such as the genetically modified micro-organ, as well as inclusion of other active agents, and pharmaceutically acceptable carriers, excipients, emollients, stabilizers, etc., as are known in the pharmaceutical industry. In some embodiments, the term “consisting essentially of” refers to a composition, whose only active ingredient is the indicated active ingredient, however, other compounds may be included which are for stabilizing, preserving, etc. the formulation, but are not involved directly in the therapeutic effect of the indicated active ingredient. In some embodiments, the term “consisting essentially of” may refer to components which facilitate the release of the active ingredient. In some embodiments, the term “consisting” refers to a composition, which contains the active ingredient and a pharmaceutically acceptable carrier or excipient.

Similarly, in some embodiments, the vector of and for use in the methods of the present invention comprise a nucleic acid sequence operably linked to one or more regulatory sequences, wherein said nucleic acid sequence encodes a therapeutic polypeptide. In another embodiment, the vector consists essentially of such a nucleic acid sequence, and in another embodiment, the vector consists of such a nucleic acid sequence.

Examples of mammals from which the micro-organs can be isolated include humans and other primates, swine, such as wholly or partially inbred swine (e.g., miniature swine, and transgenic swine), rodents, etc. Micro-organs may be processed from tissue from a variety of organs, which in one embodiment is the skin, the dermis, the lymph system, the pancreas, the liver, the gallbladder, the kidney, the digestive tract, the respiratory tract, the reproductive system, the urinary tract, the blood, the bladder, the cornea, the prostate, the bone marrow, the thymus, the spleen, or a combination thereof. Explants from these organs may comprise islet of Langerhan cells, hair follicles, glands, epithelial and connective tissue cells, or a combination thereof arranged in a microarchitecture similar to the microarchitecture of the organ from which the explant was obtained. In one embodiment, the microarchitecture of the organ from which the explant was obtained may be discerned or identified in the micro-organ explant using materials, apparati, and/or methods known in the art.

In one embodiment, the present invention provides a formulation and methods of use thereof comprising a genetically modified micro-organ. In one embodiment, the term “genetically modified micro-organ” or “GMMO” or “GMDMO” refers to a micro-organ that expresses at least one recombinant gene product. In other embodiments, reference to a micro-organ does not necessarily refer to a non-genetically modified micro-organ, but may also refer in some instances to a genetically modified micro-organ as will be clear from the context to one of skill in the art. In one embodiment, the phrase “gene product” refers to proteins, polypeptides, peptides and functional RNA molecules. In one embodiment, the gene product encoded by the nucleic acid molecule is the desired gene product to be supplied to a subject. Examples of such gene products include proteins, peptides, glycoproteins and lipoproteins normally produced by cells of the recipient subject. In one embodiment, the gene product is not naturally occurring in the organism from which the micro-organ was harvested and/or in the organism in which the GMMO is implanted, while in another embodiment, the gene product is naturally occurring. In one embodiment, the gene product of the GMMO is similar or identical to a gene product endogenously expressed by one or more cells of the micro-organ. In one embodiment, genetic modification increases the level of a gene product that would be produced in a non-genetically modified micro-organ. In another embodiment, the gene product expressed by the GMMO is not similar or identical to a gene product endogenously expressed by one or more cells of the micro-organ. In another embodiment, the gene product encoded by the nucleic acid molecule encodes a molecule that directly or indirectly controls expression of a gene of interest. In another embodiment, the gene product encoded by the nucleic acid molecule upregulates or downregulates the expression levels of the desired gene product to be supplied to a subject.

In another embodiment, genetic modification of a micro-organ may modify the expression profile of an endogenous gene. This may be achieved, for example, by introducing an enhancer, or a repressible or inducible regulatory element for controlling the expression of an endogenous gene.

Any methodology known in the art can be used for genetically altering the micro-organ explant. Any one of a number of different vectors can be used, such as viral vectors, plasmid vectors, linear DNA, etc., as known in the art, to introduce an exogenous nucleic acid fragment encoding a therapeutic agent into target cells and/or tissue. These vectors can be inserted, for example, using infection, transduction, transfection, calcium-phosphate mediated transfection, DEAE-dextran mediated transfection, electroporation, liposome-mediated transfection, biolistic gene delivery, liposomal gene delivery using fusogenic and anionic liposomes (which are an alternative to the use of cationic liposomes), direct injection, receptor-mediated uptake, magnetoporation, ultrasound, or any combination thereof, as well as other techniques known in the art (for further detail see, for example, “Methods in Enzymology” Vol. 1-317, Academic Press, Current Protocols in Molecular Biology, Ausubel F. M. et al. (eds.) Greene Publishing Associates, (1989) and in Molecular Cloning: A Laboratory Manual, 2nd Edition, Sambrook et al. Cold Spring Harbor Laboratory Press, (1989), or other standard laboratory manuals). The polynucleotide segments encoding sequences of interest can be ligated into an expression vector system suitable for transducing mammalian cells and for directing the expression of recombinant products within the transduced cells. The introduction of the exogenous nucleic acid fragment is accomplished by introducing the vector into the vicinity of the micro-organ. Once the exogenous nucleic acid fragment has been incorporated into the cells using any of the techniques described above or known in the art, the production and/or the secretion rate of the therapeutic agent encoded by the nucleic acid fragment can be quantified. In one embodiment, the term “exogenous” refers to a substance that originated outside, for example a nucleic acid that originated outside of a cell or tissue.

In one embodiment, a micro-organ of the formulation and methods of the present invention comprises a vector, which in one embodiment, facilitates recombinant gene expression. In one embodiment, the vector is a non-immunogenic gene transfer agent such as a nonviral vector (e.g. DNA plasmids or minicircle DNA), a “gutless” viral vector i.e. without endogenous genes (which in one embodiment, is due to a deletion, while in another embodiment, due to an insertion, substitution or deletion in a gene that prevents gene expression), a helper-dependent adenovirus (HDAd) vector, or adeno associated virus AAV (which in one embodiment is single stranded and in another embodiment, double stranded). In another embodiment, said formulation is so chosen such that recombinant gene expression results in lack of toxicity or immune-mediated rejection of the gene product by the micro-organ. In one embodiment, the vector is virally derived, and in another embodiment, the vector is a plasmid. In one embodiment, the virally-derived vector is derived from adenovirus, which in one embodiment, is helper-dependent adenovirus, while in another embodiment, the virally-derived vector is derived from adenovirus-associated vector, as is described hereinbelow.

In one embodiment, the term “vector” or “expression vector” refers to a carrier molecule into which a nucleic acid sequence can be inserted for introduction into a cell where it can be expressed. In one embodiment, the nucleic acid molecules are transcribed into RNA, which in some cases are then translated into a protein, polypeptide, or peptide. In other cases, RNA sequences are not translated, for example, in the production of antisense molecules or ribozymes. In one embodiment, expression vectors can contain a variety of “control sequences” which refer to nucleic acid sequences necessary for the transcription and possibly translation of an operably linked coding sequence in a particular host cell. In another embodiment, a vector further includes an origin of replication. In one embodiment the vector may be a shuttle vector, which in one embodiment can propagate both in prokaryotic and eukaryotic cells, or in another embodiment, the vector may be constructed to facilitate its integration within the genome of a cell of choice. The vector, in other embodiments may be, for example, a plasmid, a bacmid, a phagemid, a cosmid, a phage, a virus or an artificial chromosome. In one embodiment, the vector is a viral vector, which in one embodiment may be a bacteriophage, mammalian virus, or plant virus.

In one embodiment, the viral vector is an adenoviral vector. In another embodiment, the adenovirus may be of any known serotype or subgroup.

In one embodiment, some advantages of using an adenoviral vector as a gene transfer vector are: its mid-sized genome, ease of manipulation, high titer, wide target-cell range and high infectivity. Both ends of the adenoviral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off. The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNAs issued from this promoter possess a 5′-tripartite leader (TPL) sequence which makes them preferred mRNAs for translation.

In another embodiment, the adenoviral vector is a helper-dependent adenoviral vector (“HDAD”, “HD” or “HDAd” or “HD-Ad”), which in another embodiment, is synonymous with gutless, gutted, mini, fully deleted, high-capacity, Δ, or pseudo adenovirus, and which in another embodiment are deleted of all viral coding sequences except for sequences supporting DNA replication, which in one embodiment, comprise the adenovirus inverted terminal repeats and packaging sequence (ψ). In another embodiment, helper-dependent adenoviruses express no viral proteins. In one embodiment, a helper-dependent adenoviral vector comprises only the cis-acting elements of the adenovirus required to replicate and package the vector DNA. In one embodiment, a helper-dependent adenoviral vector comprises approximately 500 bp of wild-type adenovirus sequence. In another embodiment, the adenoviral vector additionally comprises stuffer DNA to meet the minimum requirement for a genome size of 27.7 kb, which in one embodiment is required for efficient packaging into the adenovirus capsid. In one embodiment, non-coding mammalian DNA, with minimal repeat sequences, is used as stuffer DNA. In another embodiment, stuffer DNA comprises non-mammalian DNA, which in one embodiment, is HPRT and/or C346 cosmid sequences.

In one embodiment, helper-dependent adenoviruses display high-efficiency in vivo transduction, high-level transgene expression, are able to maintain long-term transgene expression, in one embodiment, by avoiding chronic toxicity due to residual expression of viral proteins, or a combination thereof. In another embodiment, helper-dependent adenoviruses have high titer production, efficient infection of a broad range of cell types, the ability to infect dividing and nondividing cells, or a combination thereof. In another embodiment, a helper-dependent adenovirus for use in the methods of the instant invention does not induce a strong adaptive immune response to an implanted GM-DMO, which in one embodiment, is characterized by the generation of adeno-specific MHC class I restricted CD8 cytotoxic T lymphocytes (CTL) in immunocompetent hosts, which in one embodiment, would limit the duration of transgene expression and in another embodiment, would result in adenovirus vector clearance within several weeks. In another embodiment, a helper-dependent adenovirus for use in the methods of the instant invention does not induce high cytotoxic T cell levels (as may be measured in one embodiment by positive CD8 staining, as is known in the art), and, in another embodiment, does not induce high helper T cell levels (as may be measured in one embodiment by positive CD4 stain, as is known in the art).

In another embodiment, helper-dependent adenoviruses have a lower risk of germ line transmission and insertional mutagenesis that may cause oncogenic transformation, because the vector genome does not integrate into the host cell chromosomes. In one embodiment, the cloning capacity of helper-dependent adenoviruses is very large (in one embodiment, approximately 37 kb, in another embodiment, approximately 36 kb), allowing for the delivery of whole genomic loci, multiple transgenes, and large cis-acting elements to enhance, prolong, and regulate transgene expression.

In one embodiment, the helper-dependent adenovirus system for use with the compositions and in the methods of the present invention is similar to that described in Palmer and Ng, 2003 (Mol Ther 8:846) and in Palmer and Ng, 2004 (Mol Ther 10:792), which are hereby incorporated herein by reference in their entirety. In one embodiment, there is a stuffer sequence inserted into the E3 region of the helper virus component of the helper-dependent adenovirus system to minimize recombination between the helper adenovirus and the helper-dependent adenovirus to produce replication competent adenovirus.

In one embodiment, a vector of this invention comprises SEQ ID No. 1. In another embodiment, a vector of this invention comprises at least SEQ ID No. 1. In yet another embodiment, a vector of this invention comprises a nucleic acid sequence with at least 90% identity to SEQ ID No. 1. In still another embodiment, a vector of this invention comprises a nucleic acid sequence with at least 80% identity to SEQ ID No. 1. In a further embodiment, a vector of this invention comprises a nucleic acid sequence with at least 70% identity of SEQ ID No. 1. In one embodiment, a vector of this invention comprises SEQ ID No. 2. In one embodiment, a vector of this invention consists essentially of SEQ ID No. 2. In another embodiment, a vector of this invention comprises portions of SEQ ID No. 2. In yet another embodiment, a vector of this invention comprises a nucleic acid sequence with at least 90% identity to SEQ ID No. 2. In still another embodiment, a vector of this invention comprises a nucleic acid sequence with at least 80% identity to SEQ ID No. 2. In a further embodiment, a vector of this invention comprises a nucleic acid sequence with at least 70% identity of SEQ ID No. 2. In one embodiment, a vector of this invention comprises SEQ ID No. 3. In one embodiment, a vector of this invention consists essentially of SEQ ID No. 3. In another embodiment, a vector of this invention comprises portions of SEQ ID No. 3. In yet another embodiment, a vector of this invention comprises a nucleic acid sequence with at least 90% identity to SEQ ID No. 3. In still another embodiment, a vector of this invention comprises a nucleic acid sequence with at least 80% identity to SEQ ID No. 3. In a further embodiment, a vector of this invention comprises a nucleic acid sequence with at least 70% identity of SEQ ID No. 3. In one embodiment, a vector of this invention comprises SEQ ID No. 4. In another embodiment, a vector of this invention comprises at least SEQ ID No. 4. In yet another embodiment, a vector of this invention comprises a nucleic acid sequence with at least 90% identity to SEQ ID No. 4. In still another embodiment, a vector of this invention comprises a nucleic acid sequence with at least 80% identity to SEQ ID No. 4. In a further embodiment, a vector of this invention comprises a nucleic acid sequence with at least 70% identity of SEQ ID No. 4

In one embodiment, a vector of this invention comprises a nucleic acid sequence encoding SEQ ID No. 5. In another embodiment, a vector of this invention comprises a nucleic acid sequence encoding a portion of SEQ ID No. 5. In yet another embodiment, a vector of this invention comprises a nucleic acid sequence encoding at least SEQ ID No. 5.

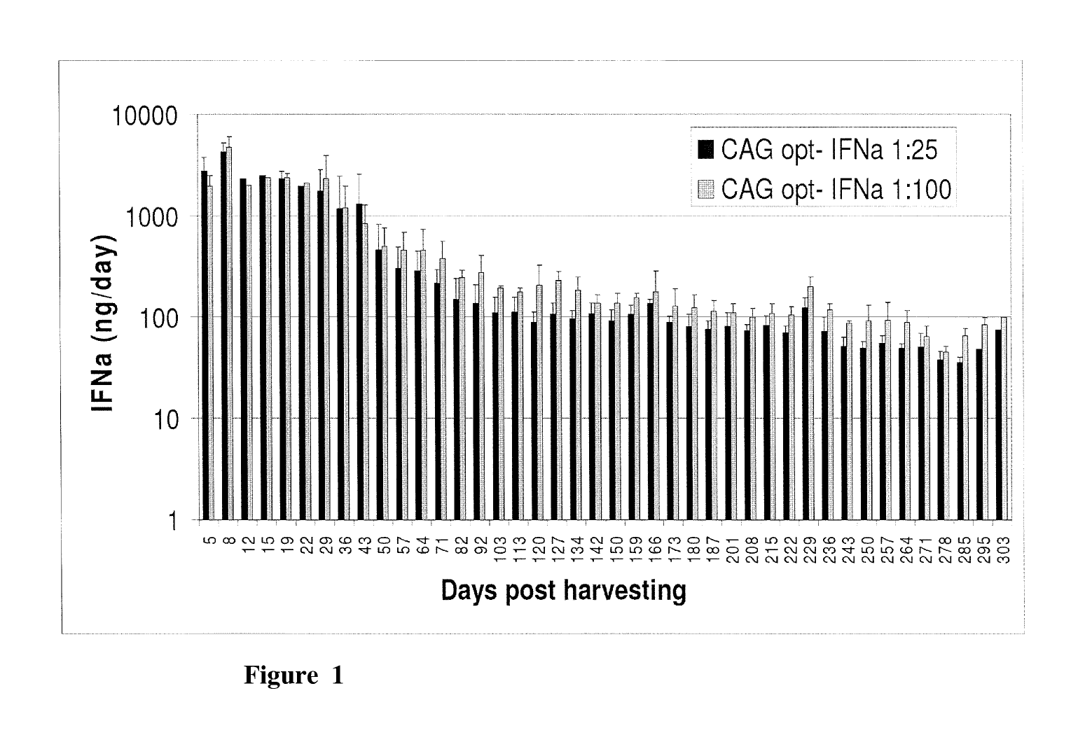

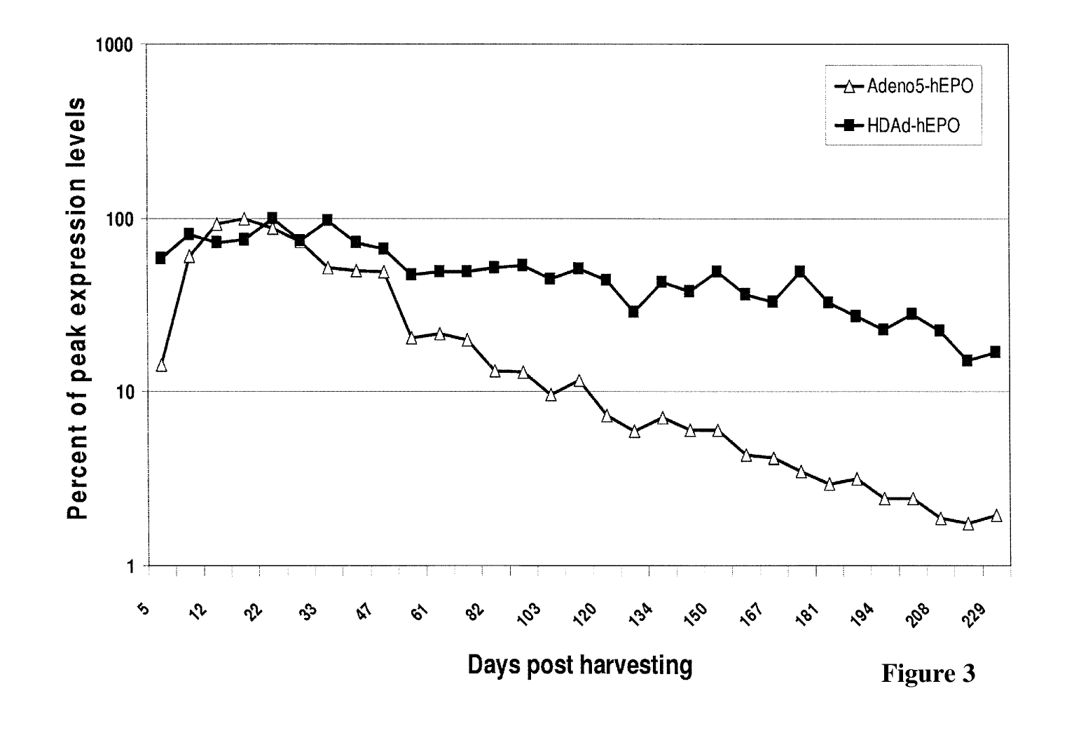

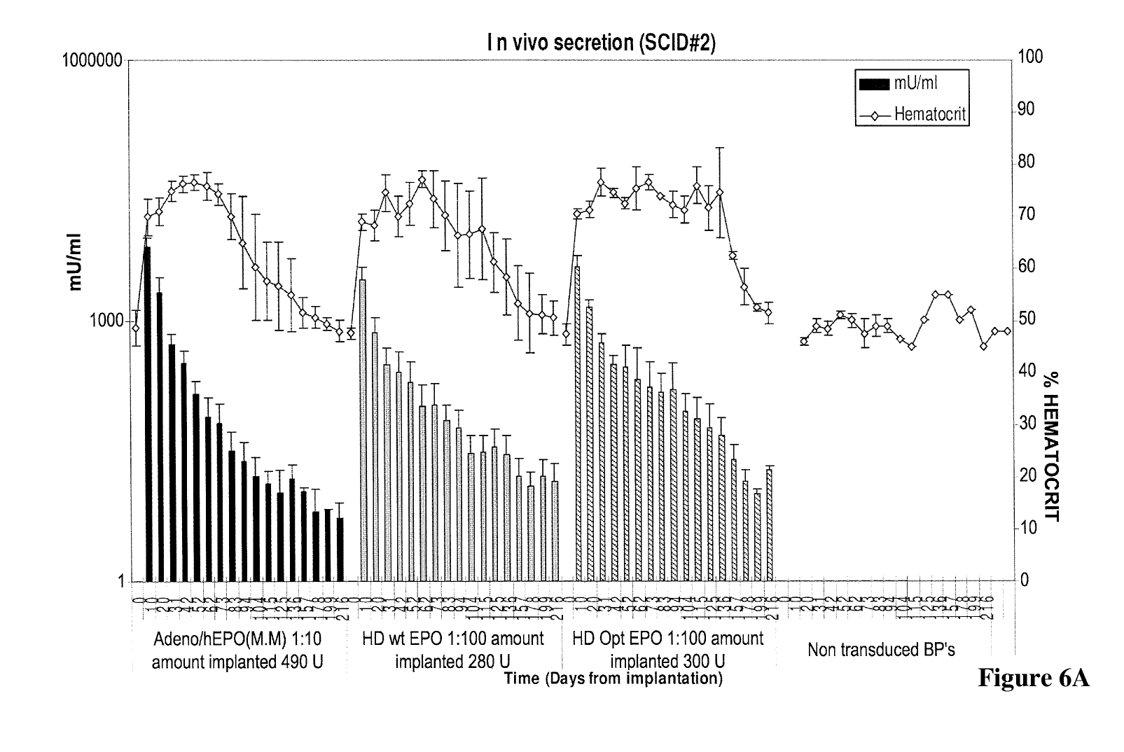

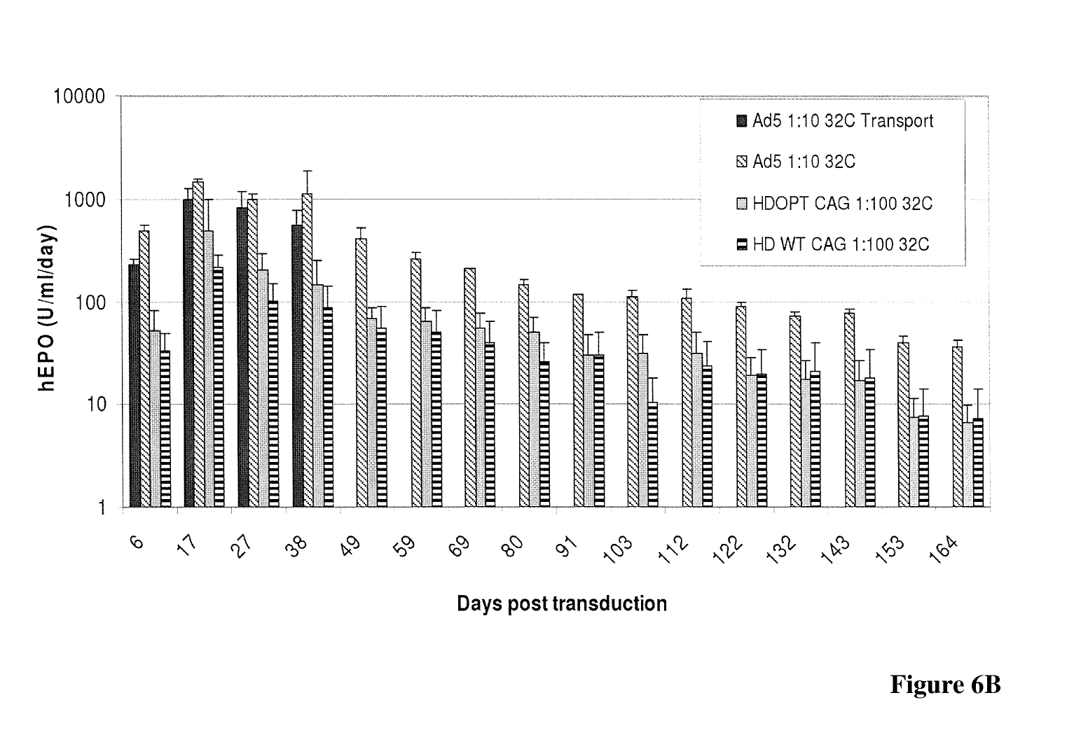

In one embodiment, formulations of the instant invention comprising helper-dependent adenoviral vectors demonstrate long-term, high in vitro (FIGS. 1, 2, and 6B) and in vivo (FIG. 6A) expression levels of EPO and IFN-alpha. In another embodiment, formulations of the instant invention comprising helper-dependent adenoviral vectors demonstrate an increased percent of peak EPO expression levels for at least 100 days post-transduction compared to micro-organs comprising adenovirus-5 (FIG. 3). Without being bound by theory, one factor that may contribute to the long-lasting, high levels of gene product from micro-organs of the instant invention is use of a helper-dependent adenovirus vector, which is non-toxic to tissue and non-immunogenic within the formulations of the present invention.

In another embodiment, the adenoviral vector is E1-deleted, while in another embodiment, the adenoviral vector additionally comprises deletions for E2, E3, E4, or a combination thereof.

In another embodiment, the viral vector is an adeno-associated viral vector (AAV). In one embodiment, AAV is a parvovirus, discovered as a contamination of adenoviral stocks. It is a ubiquitous virus (antibodies are present in 85% of the US human population) that has not been linked to any disease. It is also classified as a dependovirus, because its replication is dependent on the presence of a helper virus, such as adenovirus. At least nine serotypes have been isolated, of which AAV-2 is the best characterized. AAV may have a single-stranded linear DNA that is encapsidated into capsid proteins VP1, VP2 and VP3 to form an icosahedral virion of 20 to 24 nm in diameter.

In one embodiment, the AAV DNA is approximately 4.7 kilobases long. In one embodiment, it contains two open reading frames and is flanked by two ITRs. There are two major genes in the AAV genome: rep and cap. The rep gene codes for proteins responsible for viral replications, whereas cap codes for capsid protein VP1-3. Each ITR forms a T-shaped hairpin structure. These terminal repeats are the only essential cis components of the AAV for chromosomal integration. Therefore, in one embodiment, the AAV can be used as a vector with all viral coding sequences removed and replaced by the cassette of genes for delivery. In one embodiment, an AAV is rep negative.

In one embodiment, when using recombinant AAV (rAAV) as an expression vector, the vector comprises the 145-bp ITRs, which are only 6% of the AAV genome, which in one embodiment, leaves space in the vector to assemble a 4.5-kb DNA insertion.

In one embodiment, AAV is safe in that it is not considered pathogenic nor is it associated with any disease. The removal of viral coding sequences minimizes immune reactions to viral gene expression, and therefore, rAAV evokes only a minimal inflammatory response, if any. In another embodiment, AAV vector is double-stranded, while in another embodiment, AAV vector is self-complementary, which in one embodiment, bypasses the requirement of viral second-strand DNA synthesis, which in one embodiment, results in early transgene expression.

In another embodiment, the viral vector is a retroviral vector. The retroviruses are a group of single-stranded RNA viruses characterized by an ability to convert their RNA to double-stranded DNA in infected cells by a process of reverse-transcription. The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome.

In order to construct a retroviral vector in one embodiment, a nucleic acid encoding one or more oligonucleotide or polynucleotide sequences of interest is inserted into the viral genome in the place of certain viral sequences to produce a virus that is replication-defective. In order to produce virions, a packaging cell line containing the gag, pol, and env genes but without the LTR and packaging components is constructed. When a recombinant plasmid containing a cDNA, together with the retroviral LTR and packaging sequences is introduced into this cell line (by calcium phosphate precipitation, for example), the packaging sequence allows the RNA transcript of the recombinant plasmid to be packaged into viral particles, which are then secreted into the culture media. The media containing the recombinant retroviruses is then collected, optionally concentrated, and used for gene transfer. Retroviral vectors are able to infect a broad variety of cell types. However, integration and stable expression require the division of host cells.

In other embodiments, the viral vector is derived from a virus such as vaccinia virus, lentivirus, polio virus, hepatitis virus, papilloma virus, cytomegalovirus, simian virus, or herpes simplex virus.

In certain embodiments of the invention, the vector comprising a nucleic acid sequence may comprise naked recombinant DNA or plasmids. Transfer of the construct may be performed by any method which physically or chemically permeabilizes the cell membrane. In one embodiment, the vector is a mini-circle DNA, which in one embodiment, is a supercoiled DNA molecule for non-viral gene transfer, which has neither a bacterial origin of replication nor an antibiotic resistance marker. In another embodiment, mini-circle DNA comprises no bacterial control regions from gene delivery vectors during the process of plasmid production. They are thus smaller and potentially safer than other plasmids used in gene therapy. In one embodiment, mini-circle DNA produce high yield, are simple to purify, and provide robust and persistent transgene expression.

Construction of vectors using standard recombinant techniques is well known in the art (see, for example, Maniatis, et al., Molecular Cloning, A Laboratory Manual (Cold Spring Harbor, 1990) and Ausubel, et al., 1994, Current Protocols in Molecular Biology (John Wiley & Sons, 1996), both incorporated herein by reference).

In another embodiment, a vector further comprises an insertion of a heterologous nucleic acid sequence encoding a marker polypeptide. The marker polypeptide may comprise, for example, yECitrine, green fluorescent protein (GFP/EGFP), DS-Red (red fluorescent protein), secreted alkaline phosphatase (SEAP), β-galactosidase, chloramphenicol acetyl transferase, luciferase, or any number of other reporter proteins known to one skilled in the art.

In another embodiment, the vectors may comprise one or more genes of interest. Thus, in one embodiment, a vector of the instant invention may comprise a gene of interest, which in one embodiment, is erythropoietin or interferon alpha2b, which in one embodiment, expresses a marker, and in another embodiment, is linked in frame to a marker, which in one embodiment allows identification of the gene product of interest and in another embodiment, allows the distinction between a gene product of interest produced by a micro-organ and a similar gene product produced endogenously by host cells outside of the micro-organ(s).

In one embodiment, a vector comprising a nucleic acid encoding a therapeutic polypeptide of the instant invention is introduced into a micro-organ. There are a number of techniques known in the art for introducing cassettes and/or vectors into cells, for affecting the methods of the present invention, such as, but not limited to: direct DNA uptake techniques, and virus, plasmid, linear DNA or liposome mediated transduction, receptor-mediated uptake and magnetoporation methods employing calcium-phosphate mediated and DEAE-dextran mediated methods of introduction, electroporation or liposome-mediated transfection, (for further detail see, for example, “Methods in Enzymology” Vol. 1-317, Academic Press, Current Protocols in Molecular Biology, Ausubel F. M. et al. (eds.) Greene Publishing Associates, (1989) and in Molecular Cloning: A Laboratory Manual, 2nd Edition, Sambrook et al. Cold Spring Harbor Laboratory Press, (1989), or other standard laboratory manuals).

In one embodiment, bombardment with nucleic acid coated particles may be a method for transferring a naked DNA expression construct into cells. This method depends on the ability to accelerate DNA-coated micro-projectiles to a high velocity allowing them to pierce cell membranes and enter cells without killing them. Several devices for accelerating small particles have been developed. One such device relies on a high voltage discharge to generate an electrical current, which in turn provides the motive force. The micro-projectiles used have comprised biologically inert or biocompatible substances such as tungsten or gold beads. It is to be understood that any of these methods may be utilized for introduction of the desired sequences into cells, and cells thereby produced are to be considered as part of this invention, as is their use for effecting the methods of this invention.

In one embodiment, the vectors of the formulations and methods of the instant invention comprise a nucleic acid sequence. As used herein, the term “nucleic acid” refers to polynucleotide or to oligonucleotides such as deoxyribonucleic acid (DNA), and, where appropriate, ribonucleic acid (RNA) or mimetic thereof. The term should also be understood to include, as equivalents, analogs of RNA or DNA made from nucleotide analogs, and, as applicable to the embodiment being described, single (sense or antisense) and double-stranded polynucleotide. This term includes oligonucleotides composed of naturally occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

In one embodiment, the term “nucleic acid” or “oligonucleotide” refers to a molecule, which may include, but is not limited to, prokaryotic sequences, eukaryotic mRNA, cDNA from eukaryotic mRNA, genomic DNA sequences from eukaryotic (e.g., mammalian) DNA, and even synthetic DNA sequences. The term also refers to sequences that include any of the known base analogs of DNA and RNA.

The nucleic acids can be produced by any synthetic or recombinant process, which are well known in the art. Nucleic acids can further be modified to alter biophysical or biological properties by means of techniques known in the art. For example, the nucleic acid can be modified to increase its stability against nucleases (e.g., “end-capping”), or to modify its solubility, or binding affinity to complementary sequences. These nucleic acids may comprise the vector, the expression cassette, the promoter sequence, the gene of interest, or any combination thereof. In another embodiment, its lipophilicity may be modified, which, in turn, will reflect changes in the systems employed for its delivery, and in one embodiment, may further be influenced by whether such sequences are desired for retention within, or permeation through the skin, or any of its layers. Such considerations may influence any compound used in this invention, in the methods and systems described.

In one embodiment, DNA can be synthesized chemically from the four nucleotides in whole or in part by methods known in the art. Such methods include those described in Caruthers (1985; Science 230:281-285). DNA can also be synthesized by preparing overlapping double-stranded oligonucleotides, filling in the gaps, and ligating the ends together (see, generally, Sambrook et al. (1989; Molecular Cloning—A Laboratory Manual, 2nd Edition. Cold Spring Habour Laboratory Press, New York)). In another embodiment, inactivating mutations may be prepared from wild-type DNA by site-directed mutagenesis (see, for example, Zoller et al. (1982; DNA. 1984 December; 3(6):479-88); Zoller (1983); and Zoller (1984; DNA. 1984 December; 3(6):479-88); McPherson (1991; Directed Mutagenesis: A Practical Approach. Oxford University Press, NY)). The DNA obtained can be amplified by methods known in the art. One suitable method is the polymerase chain reaction (PCR) method described in Saiki et al. (1988; Science. 1988 Jan. 29; 239(4839):487-491), Mullis et al., U.S. Pat. No. 4,683,195, and Sambrook et al. (1989).

Methods for modifying nucleic acids to achieve specific purposes are disclosed in the art, for example, in Sambrook et al. (1989). Moreover, the nucleic acid sequences of the invention can include one or more portions of nucleotide sequence that are non-coding for the protein of interest. Variations in DNA sequences, which are caused by point mutations or by induced modifications (including insertion, deletion, and substitution) to enhance the activity, half-life or production of the polypeptides encoded thereby, are also encompassed in the invention.

The formulations of this invention may comprise nucleic acids, in one embodiment, or in another embodiment, the methods of this invention may include delivery of the same, wherein, in another embodiment, the nucleic acid is a part of a vector.

The efficacy of a particular expression vector system and method of introducing nucleic acid into a cell can be assessed by standard approaches routinely used in the art as described hereinbelow.

As will be appreciated by one skilled in the art, a fragment or derivative of a nucleic acid sequence or gene that encodes for a protein or peptide can still function in the same manner as the entire wild type gene or sequence. Likewise, forms of nucleic acid sequences can have variations as compared to wild type sequences, nevertheless encoding the protein or peptide of interest, or fragments thereof, retaining wild type function exhibiting the same biological effect, despite these variations. Each of these represents a separate embodiment of this present invention.

In one embodiment, the formulations and methods of the present invention may be used for gene silencing applications. In one embodiment, the activity or function of a particular gene is suppressed or diminished, via the use of anti-sense oligonucleotides, which are chimeric molecules, containing two or more chemically distinct regions, each made up of at least one nucleotide.

In one embodiment, chimeric oligonucleotides comprise at least one region wherein the oligonucleotide is modified so as to confer upon the oligonucleotide an increased resistance to nuclease degradation, increased cellular uptake, and/or increased binding affinity for the target polynucleotide. An additional region of the oligonucleotide may serve as a substrate for enzymes capable of cleaving RNA:DNA or RNA:RNA hybrids, which according to this aspect of the invention, serves as a means of gene silencing via degradation of specific sequences. Cleavage of the RNA target can be routinely detected by gel electrophoresis and, if necessary, associated nucleic acid hybridization techniques known in the art.

The chimeric antisense oligonucleotides may, in one embodiment, be formed as composite structures of two or more oligonucleotides and/or modified oligonucleotides, as is described in the art (see, for example, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922), and may, in another embodiment, comprise a ribozyme sequence.

Inhibition of gene expression, activity or function is effected, in another embodiment, via the use of small interfering RNAs, which provides sequence-specific inhibition of gene expression. Administration of double stranded/duplex RNA (dsRNA) corresponding to a single gene in an organism can silence expression of the specific gene by rapid degradation of the mRNA in affected cells. This process is referred to as gene silencing, with the dsRNA functioning as a specific RNA inhibitor (RNAi). RNAi may be derived from natural sources, such as in endogenous virus and transposon activity, or it can be artificially introduced into cells (Elbashir S M, et al (2001). Nature 411:494-498) via microinjection (Fire et al. (1998) Nature 391: 806-11), or by transformation with gene constructs generating complementary RNAs or fold-back RNA, or by other vectors (Waterhouse, P. M., et al. (1998). Proc. Natl. Acad. Sci. USA 95, 13959-13964 and Wang, Z., et al. (2000). J. Biol. Chem. 275, 40174-40179). The RNAi mediating mRNA degradation, in one embodiment, comprises duplex or double-stranded RNA, or, in other embodiments, include single-stranded RNA, isolated RNA (partially purified RNA, essentially pure RNA, synthetic RNA, recombinantly produced RNA), as well as altered RNA that differs from naturally occurring RNA by the addition, deletion and/or alteration of one or more nucleotides.

In one embodiment, the nucleic acid of the formulations and methods of the instant invention encode a therapeutic polypeptide. In one embodiment, the term “polypeptide” refers to a molecule comprised of amino acid residues joined by peptide (i.e., amide) bonds and includes peptides, polypeptides, and proteins. Hence, in one embodiment, the polypeptides of this invention may have single or multiple chains of covalently linked amino acids and may further contain intrachain or interchain linkages comprised of disulfide bonds. In one embodiment, some polypeptides may also form a subunit of a multiunit macromolecular complex. In one embodiment, the polypeptides can be expected to possess conformational preferences and to exhibit a three-dimensional structure. Both the conformational preferences and the three-dimensional structure will usually be defined by the polypeptide's primary (i.e., amino acid) sequence and/or the presence (or absence) of disulfide bonds or other covalent or non-covalent intrachain or interchain interactions.

In one embodiment, the term “peptide” refers to native peptides (either degradation products, synthetically synthesized peptides or recombinant peptides).

In one embodiment, the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” may include both D- and L-amino acids.

As used herein, the term “amino acid” refers to either the D or L stereoisomer form of the amino acid, unless otherwise specifically designated. Also encompassed within the scope of this invention are equivalent proteins or equivalent peptides, e.g., having the biological activity of purified wild type IFN. “Equivalent proteins” and “equivalent polypeptides” refer to compounds that depart from the linear sequence of the naturally occurring proteins or polypeptides, but which have amino acid substitutions that do not change it's biologically activity. These equivalents can differ from the native sequences by the replacement of one or more amino acids with related amino acids, for example, similarly charged amino acids, or the substitution or modification of side chains or functional groups.

The peptides or polypeptides, or the DNA sequences encoding same, may be obtained from a variety of natural or unnatural sources, such as a prokaryotic or a eukaryotic cell. In one embodiment, the source cell may be wild type, recombinant, or mutant. In another embodiment, the plurality of peptides or polypeptides may be endogenous to microorganisms, such as bacteria, yeast, or fungi, to a virus, to an animal (including mammals, invertebrates, reptiles, birds, and insects) or to a plant cell.

In another embodiment, the peptides or polypeptides may be obtained from more specific sources, such as the surface coat of a virion particle, a particular cell lysate, a tissue extract, or they may be restricted to those polypeptides that are expressed on the surface of a cell membrane.

In another embodiment, the peptide or polypeptide is derived from a particular cell or tissue type, developmental stage or disease condition or stage. In one embodiment, the disease condition or stage is cancer, in another embodiment, the disease condition is an infection, which in another embodiment, is an HIV infection. In another embodiment, the disease condition is a developmental disorder, while in another embodiment, the disease condition is a metabolic disorder.

The polypeptide of the present invention can be of any size. As can be expected, the polypeptides can exhibit a wide variety of molecular weights, some exceeding 150 to 200 kilodaltons (kD). Typically, the polypeptides may have a molecular weight ranging from about 5,000 to about 100,000 daltons. Still others may fall in a narrower range, for example, about 10,000 to about 75,000 daltons, or about 20,000 to about 50,000 daltons. In an alternative embodiment, the polypeptides of the present invention may be 1-250 amino acid residues long. In another embodiment, the polypeptides of the present invention may be 10-200 amino acid residues long. In an alternative embodiment, the polypeptides of the present invention may be 50-100 amino acid residues long. In an alternative embodiment, the polypeptides of the present invention may be 1-250 amino acid residues long. In an alternative embodiment, the polypeptides of the present invention may be 1-250 amino acid residues long. In one embodiment, the maximum size of the peptide or polypeptide is determined by the vector from which it is expressed, which in one embodiment, is approximately between 20 and 37 kD, between 20 and 25 kD, between 25 and 30 kD, between 30 and 37 kD, or between 35 and 37 kD. In another embodiment, the polypeptide is a 34 kD glycoprotein.

According to other embodiments of the present invention, recombinant gene products may be encoded by a polynucleotide having a modified nucleotide sequence, as compared to a corresponding natural polynucleotide.

As described hereinabove, in one embodiment, the formulations and methods of the present invention provide a therapeutic formulation comprising a nucleic acid sequence encoding a therapeutic polypeptide. In one embodiment, the term “therapeutic” refers to a molecule, which when provided to a subject in need, provides a beneficial effect. In some cases, the molecule is therapeutic in that it functions to replace an absence or diminished presence of such a molecule in a subject. In one embodiment, the therapeutic protein is that of a protein which is absent in a subject, such as in cases of subjects with an endogenous null or mis-sense mutation of a required protein. In other embodiments, the endogenous protein is mutated, and produces a non-functional protein, compensated for by the provision of the functional protein. In other embodiments, expression of a heterologous protein is additive to low endogenous levels, resulting in cumulative enhanced expression of a given protein. In other embodiments, the molecule stimulates a signaling cascade that provides for expression, or secretion, or others of a critical element for cellular or host functioning.

In one embodiment, the term “therapeutic formulation” describes a substance applicable for use in the diagnosis, or in another embodiment, cure, or in another embodiment, mitigation, or in another embodiment, treatment, or in another embodiment, prevention of a disease, disorder, condition or infection. In one embodiment, the “therapeutic formulation” of this invention refers to any substance which affect the structure or function of the target to which it is applied.

In another embodiment, the “therapeutic formulation” of the present invention is a molecule that alleviates a symptom of a disease or disorder when administered to a subject afflicted thereof. In one embodiment, the “therapeutic formulation” of this invention is a synthetic molecule, or in another embodiment, a naturally occurring compound isolated from a source found in nature.

In one embodiment, a “therapeutic formulation” of the present invention may alleviate symptoms of hepatitis. In one embodiment, a therapeutic formulation may alleviate symptoms of hepatitis C. In another embodiment, a therapeutic formulation may alleviate symptoms of hepatitis B. In yet another embodiment, a therapeutic formulation may alleviate symptoms of hepatitis D. In one embodiment, the disease or disorder is hepatitis C, genotype 1. In another embodiment, the disease or disorder is hepatitis C, genotype 2. In yet another embodiment, the disease or disorder is hepatitis C, genotype 3. In still another embodiment, the disease or disorder is hepatitis C, having a combination of genotypes.

In one embodiment, the therapeutic polypeptide is erythropoietin, while in another embodiment, the therapeutic polypeptide is interferon alpha, which in one embodiment, is interferon alpha 2b. In one embodiment, said therapeutic polypeptide is any other therapeutic polypeptide.

In one embodiment, “treatment” refers to both therapeutic treatment and prophylactic or preventative measures, wherein the object is to prevent or lessen the targeted pathologic condition or disorder as described hereinabove. Thus, in one embodiment, treating may include directly affecting or curing, suppressing, inhibiting, preventing, reducing the severity of, delaying the onset of, reducing symptoms associated with the disease, disorder or condition, or a combination thereof. Thus, in one embodiment, “treating” refers inter alia to delaying progression, expediting remission, inducing remission, augmenting remission, speeding recovery, increasing efficacy of or decreasing resistance to alternative therapeutics, or a combination thereof. In one embodiment, “preventing” refers, inter alia, to delaying the onset of symptoms, preventing relapse to a disease, decreasing the number or frequency of relapse episodes, increasing latency between symptomatic episodes, or a combination thereof. In one embodiment, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

In one embodiment, symptoms are primary, while in another embodiment, symptoms are secondary. In one embodiment, “primary” refers to a symptom that is a direct result of a particular disease, while in one embodiment; “secondary” refers to a symptom that is derived from or consequent to a primary cause. In one embodiment, the compounds for use in the present invention treat primary or secondary symptoms or secondary complications related to said disease. In another embodiment, “symptoms” may be any manifestation of a disease or pathological condition.

In one embodiment, a therapeutic nucleic acid may encode a therapeutic polypeptide, which may in one embodiment, comprise an enzyme, an enzyme cofactor, a cytotoxic protein, an antibody, a channel protein, a transporter protein, a growth factor, a hormone, a cytokine, a receptor, a mucin, a surfactant, an aptamer or a hormone. In another embodiment, the therapeutic polypeptide may be of one or more of the categories as described above. In another embodiment, a therapeutic nucleic acid may encode functional RNA as described hereinbelow. A detailed description of therapeutic nucleic acids and polypeptides encoded by therapeutic nucleic acids of this invention, may be found in US-2012-0003196-A1, which is incorporated herein by reference in it entirety.

In one embodiment, the “therapeutic formulation” of this invention is antibacterial, antiviral, antifungal or antiparasitic. In another embodiment, the therapeutic formulation has cytotoxic or anti-cancer activity. In another embodiment, the therapeutic formulation is immunostimulatory. In another embodiment, the therapeutic formulation inhibits inflammatory or immune responses.

In one embodiment, the therapeutic nucleic acids may encode or the therapeutic polypeptides may be cytokines, such as interferons or interleukins, or their receptors. Lack of expression of cytokines, or of the appropriate ones, has been implicated in susceptibility to diseases, and enhanced expression may lead to resistance to a number of infections. Expression patterns of cytokines may be altered to produce a beneficial effect, such as for example, a biasing of the immune response toward a Th1 type expression pattern, or a Th2 pattern in infection, or in autoimmune disease, wherein altered expression patterns may prove beneficial to the host.

In one embodiment, the therapeutic nucleic acid of the present invention may encode or the therapeutic polypeptide may be an immunomodulating protein. In one embodiment, the immunomodulating protein comprises cytokines, chemokines, complement or components, such as interleukins 1 to 15, interferons alpha, beta or gamma, tumour necrosis factor, granulocyte-macrophage colony stimulating factor (GM-CSF), macrophage colony stimulating factor (M-CSF), granulocyte colony stimulating factor (G-CSF), chemokines such as neutrophil activating protein (NAP), macrophage chemoattractant and activating factor (MCAF), RANTES, macrophage inflammatory peptides MIP-1a and MIP-1b, or complement components.

In another embodiment, the gene comprises a reporter gene. In one embodiment, the reporter gene encodes a fluorescent protein. In one embodiment, the fluorescent protein is yECitrine or a yellow fluorescent protein. In one embodiment, the fluorescent protein is the jellyfish green fluorescent protein, or a mutant or variant thereof. In another embodiment, the GMMOs specifically may comprise any gene other than a reporter gene or a gene encoding a reporter protein.

In another embodiment, the reporter gene confers drug resistance. In one embodiment, the reporter gene confers resistance to an antibiotic, such as, for example, ampicilin, kanamycin, tetracycline, or others, as will be appreciated by one skilled in the art. In another embodiment, the antibiotic resistance genes may include those conferring resistance to neomycin (neo), blasticidin, spectinomycin, erythromycin, phleomycin, Tn917, gentamycin, and bleomycin. An example of the neomycin resistance gene is the neomycin resistance gene of transposon Tn5 that encodes for neomycin phosphotransferase 11, which confers resistance to various antibiotics, including G418 and kanamycin. In another embodiment, the reporter is a chloramphenicol acetyl transferase gene (cat) and confers resistance to chloramphenicol.

In one embodiment, the formulations and methods of this invention are for prevention of, or therapeutic intervention of viral infection, or in another embodiment, bacterial, parasitic, or fungal infection, or a combination thereof. In one embodiment, viral infection is a hepatitis viral infection. In one embodiment, the hepatitis viral infection is a hepatitis B infection. In another embodiment, the hepatitis viral infection is a hepatitis C infection. In one embodiment, the hepatitis C infection is a hepatitis C genotype 1 infection. In another embodiment, the hepatitis C infection is a hepatitis C genotype 2 infection. In yet another embodiment, the hepatitis C infection is a hepatitis C genotype 3 infection. In one embodiment, the hepatitis C infection is a hepatitis C genotype 4 infection. In another embodiment, the hepatitis C infection is a hepatitis C genotype 5 infection. In yet another embodiment, the hepatitis C infection is a hepatitis C genotype 6 infection. In still another embodiment, the hepatitis C infection is a hepatitis C genotype 7 infection. In a further embodiment, the hepatitis C infection is a hepatitis C genotype 8 infection. In another embodiment, the hepatitis C infection is a hepatitis C genotype 9 infection. In yet another embodiment, the hepatitis C infection is a hepatitis C genotype 10 infection. In still another embodiment, the hepatitis C infection is a hepatitis C genotype 11 infection. In another embodiment, the hepatitis C infection is caused by a combination of hepatitis C viral genotypes. In a further embodiment, the hepatitis viral infection is a hepatitis D infection. In another embodiment, the hepatitis viral infection is caused by a combination of hepatitis B and hepatitis D infection.

According to this aspect of the invention, the formulations and methods of this invention are for prevention of, or therapeutic intervention in disease. A detailed description of formulation and methods of this invention can be found in US-2012-0003196-A1, which is incorporated herein by reference in its entirety.

In one embodiment, the formulations and methods of the instant invention comprise at least one genetically modified micro-organ that expresses and secretes interferon, said genetically modified micro-organ comprising a micro-organ transduced with a helper-dependent adenoviral vector, said vector comprising a nucleic acid sequence encoding an interferon operably linked to one or more regulatory sequences, wherein said nucleic acid encoding interferon comprises SEQ. ID. No. 1. In another embodiment, formulations and methods of the instant invention comprise at least one genetically modified micro-organ that expresses and secretes interferon, said genetically modified micro-organ comprising a helper dependent adenovirus vector comprising a nucleic acid sequence encoding interferon operably linked to one or more regulatory sequences.

In one embodiment, a nucleic acid molecule introduced into a cell of a micro-organ is in a form suitable for expression in the cell of the gene product encoded by the nucleic acid. Accordingly, in one embodiment, the nucleic acid molecule includes coding and regulatory sequences required for transcription of a gene (or portion thereof). When the gene product is a protein or peptide, the nucleic acid molecule includes coding and regulatory sequences required for translation of the nucleic acid molecule include promoters, enhancers, polyadenylation signals, sequences necessary for transport of an encoded protein or peptide, for example N-terminal signal sequences for transport of proteins or peptides to the surface of the cell or secretion, in one embodiment.

Nucleotide sequences which regulate expression of a gene product (e.g., promoter and enhancer sequences) are selected based upon the type of cell in which the gene product is to be expressed and the desired level of expression of the gene product. For example, a promoter known to confer cell-type specific expression of a gene linked to the promoter can be used. A promoter specific for myoblast gene expression can be linked to a gene of interest to confer muscle-specific expression of that gene product. Muscle-specific regulatory elements which are known in the art include upstream regions from the dystrophin gene (Klamut et al., (1989) Mol. Cell Biol. 9:2396), the creatine kinase gene (Buskin and Hauschka, (1989) Mol. Cell Biol. 9:2627) and the troponin gene (Mar and Ordahl, (1988) Proc. Natl. Acad. Sci. USA. 85:6404). Negative response elements in keratin genes mediate transcriptional repression (Jho Sh et al, (2001). J. Biol Chem). Regulatory elements specific for other cell types are known in the art (e.g., the albumin enhancer for liver-specific expression; insulin regulatory elements for pancreatic islet cell-specific expression; various neural cell-specific regulatory elements, including neural dystrophin, neural enolase and A4 amyloid promoters). Alternatively, a regulatory element which can direct constitutive expression of a gene in a variety of different cell types, such as a viral regulatory element, can be used. Examples of viral promoters commonly used to drive gene expression include those derived from polyoma virus, Adenovirus 2, cytomegalovirus (CMV) and Simian Virus 40, and retroviral LTRs. Alternatively, a regulatory element which provides inducible expression of a gene linked thereto can be used. The use of an inducible regulatory element (e.g., an inducible promoter) allows for modulation of the production of the gene product in the cell. Examples of potentially useful inducible regulatory systems for use in eukaryotic cells include hormone-regulated elements (e.g., see Mader, S, and White, J. H. (1993) Proc. Natl. Acad. Sci. USA 90:5603-5607), synthetic ligand-regulated elements (see, e.g., Spencer, D. M. et al 1993) Science 262:1019-1024) and ionizing radiation-regulated elements (e.g., see Manome, Y. Et al. (1993) Biochemistry 32:10607-10613; Datta, R. et al. (1992) Proc. Natl. Acad. Sci. USA 89:1014-10153). Additional tissue-specific or inducible regulatory systems which may be developed can also be used in accordance with the invention.

In one embodiment, a regulatory sequence of the instant invention may comprise a CMV promoter, while in another embodiment; the regulatory sequence may comprise a CAG promoter. In one embodiment, a CAG promoter is a composite promoter that combines the human cytomegalovirus immediate-early enhancer and a modified chicken beta-actin promoter and first intron. In one embodiment, a regulatory sequence may comprise a simian virus (SV)-40 polyadenylation sequence, which in one embodiment, is the mechanism by which most messenger RNA molecules are terminated at their 3′ ends in eukaryotes. In one embodiment, the polyadenosine (poly-A) tail protects the mRNA molecule from exonucleases and is important for transcription termination, for export of the mRNA from the nucleus, and for translation. In another embodiment, a formulation of the present invention may comprise one or more regulatory sequences.

In one embodiment, formulations of the instant invention comprising CMV or CAG promoters in conjunction with SV40 demonstrate long-term, high in vitro (FIGS. 1, 5, and 7) and in vivo (FIG. 6A) expression levels of EPO and IFN-alpha. Without being bound by theory, one factor that may contribute to the long-lasting, high levels of gene product from micro-organs of the instant invention is the use of CMV, or alternatively, CAG as a promoter, which may be especially effective in micro-organ explants in promoting constitutive gene expression.

In one embodiment, the term “promoter” refers to a DNA sequence, which, in one embodiment, is directly upstream of the coding sequence and is important for basal and/or regulated transcription of a gene. In one embodiment, a promoter of the present invention is operatively linked to a gene of interest. In another embodiment, the promoter is a mutant of the endogenous promoter, which is normally associated with expression of the gene of interest, under the appropriate conditions.

In one embodiment, a promoter of the compositions and for use in the methods of the present invention is a regulatable promoter. In another embodiment, a regulatable promoter refers to a promoter whereby expression of a gene downstream occurs as a function of the occurrence or provision of specific conditions which stimulate expression from the particular promoter. In some embodiments, such conditions result in directly turning on expression, or in other embodiments, remove impediments to expression. In some embodiments, such conditions result in turning off, or reducing expression.

In one embodiment, such conditions may comprise specific temperatures, nutrients, absence of nutrients, presence of metals, or other stimuli or environmental factors as will be known to one skilled in the art. In one embodiment, a regulatable promoter may be regulated by galactose (e.g. UDP-galactose epimerase (GAL10), galactokinase (GAL1)), glucose (e.g. alcohol dehydrogenase II (ADH2)), or phosphate (e.g. acid phosphatase (PHO5)). In another embodiment, a regulatable promoter may be activated by heat shock (heat shock promoter) or chemicals such as IPTG or Tetracycline, or others, as will be known to one skilled in the art. It is to be understood that any regulatable promoter, and conditions for such regulation is encompassed by the vectors, nucleic acids and methods of this invention, and represents an embodiment thereof.

In one embodiment, the formulations and methods of the instant invention increase the levels of a therapeutic polypeptide or nucleic acid by at least 5% over basal levels. In another embodiment, the levels of a therapeutic polypeptide or nucleic acid are increased by at least 7%, in another embodiment, by at least 10%, in another embodiment, by at least 15%, in another embodiment, by at least 20%, in another embodiment, by at least 25%, in another embodiment, by at least 30%, in another embodiment, by at least 40%, in another embodiment, by at least 50%, in another embodiment, by at least 60%, in another embodiment, by at least 75%, in another embodiment, by at least 100%, in another embodiment, by at least 125%, in another embodiment, by at least 150% over basal levels, in another embodiment, by at least 200% over basal levels. In one embodiment, the therapeutic polypeptide is an interferon and the nucleic acid encoding the therapeutic polypeptide encodes interferon. In one embodiment the interferon is interferon alpha. In another embodiment, the interferon is alpha 2a or alpha 2b. In yet another embodiment, the interferon is encoded by SEQ ID No. 1. In still another embodiment, the interferon is encoded by SEQ ID No. 4. In a further embodiment the therapeutic polypeptide comprises SEQ ID No. 5.

In one embodiment, expression of a therapeutic polypeptide or nucleic acid via the formulation of the present invention is increased compared to “basal levels”, which in one embodiment, are levels of the gene expressed in hosts or cell culture that had not been administered or otherwise contacted with the therapeutic formulation of the present invention.

In another embodiment, the formulations and methods of the instant invention increase the levels of a therapeutic polypeptide or nucleic acid to approximately 2000 ng/day, or in another embodiment, 1500 ng/day, or in another embodiment, 1000 ng/day, or in another embodiment, 750 ng/day, or in another embodiment, 500 ng/day, or in another embodiment, 250 ng/day, or in another embodiment, 150 ng/day, or in another embodiment, 100 ng/day, or in another embodiment, 75 ng/day, or in another embodiment, 50 ng/day, or in another embodiment, 25 ng/day. In another embodiment, the formulations and methods of the instant invention increase the levels of a therapeutic polypeptide to between 20-70 mU/mL, or in another embodiment, 50-100 mU/mL, or in another embodiment, 5-20 mU/mL, or in another embodiment, 100-200 mU/mL, or in another embodiment, 10-70 mU/mL, or in another embodiment, 5-80 mU/mL. In another embodiment, the formulations and methods of the instant invention increase the levels of a therapeutic polypeptide to between 500-1000 mU/mL, or in another embodiment, 250-750 mU/mL, or in another embodiment, 500-5000 mU/mL.

In one embodiment, the formulations and methods of the instant invention increase the levels of a functional marker, which in one embodiment, is hematocrit levels, by at least 5% over basal levels. In another embodiment, the levels of the functional marker are increased by at least 7%, in another embodiment, by at least 10%, in another embodiment, by at least 15%, in another embodiment, by at least 20%, in another embodiment, by at least 25%, in another embodiment, by at least 30%, in another embodiment, by at least 40%, in another embodiment, by at least 50%, in another embodiment, by at least 60%, in another embodiment, by at least 75%, in another embodiment, by at least 100%, in another embodiment, by at least 125%, in another embodiment, by at least 150% over basal levels, in another embodiment, by at least 200% over basal levels.

In one embodiment, the therapeutic formulation of the present invention is “long-lasting”, which in one embodiment refers to a formulation that can increase secretion, expression, production, circulation or persistence of a therapeutic polypeptide or nucleic acid. In one embodiment, expression levels of a therapeutic polypeptide or nucleic acid are increased over basal levels for at least one month, or in another embodiment, for at least six months. In another embodiment, the levels of hematocrit are increased for at least 2 weeks, in another embodiment, for at least 3 weeks, in another embodiment, for at least 4 weeks, in another embodiment, for at least 5 weeks, in another embodiment, for at least 6 weeks, in another embodiment, for at least 8 weeks, in another embodiment, for at least 2 months, in another embodiment, for at least 2 months in another embodiment, for at least 2 months in another embodiment, for at least 3 months in another embodiment, for at least 4 months, in another embodiment, for at least 5 months, in another embodiment, for at least 7 months, in another embodiment, for at least 8 months, in another embodiment, for at least 9 months, in another embodiment, for at least 10 months, in another embodiment, for at least 11 months, or, in another embodiment, for at least 1 year. In another embodiment, expression levels of a therapeutic polypeptide or nucleic acid are increased for at least 4-6 months.

In one embodiment, the nucleic acid sequence encoding a therapeutic polypeptide or nucleic acid is optimized for increased levels of therapeutic polypeptide or nucleic acid expression, or, in another embodiment, for increased duration of therapeutic polypeptide or nucleic acid expression, or, in another embodiment, a combination thereof.

In one embodiment, the term “optimized” refers to a desired change, which, in one embodiment, is a change in gene expression and, in another embodiment, in protein expression. In one embodiment, optimized gene expression is optimized regulation of gene expression. In another embodiment, optimized gene expression is an increase in gene expression. According to this aspect and in one embodiment, a 2-fold through 1000-fold increase in gene expression compared to wild-type is contemplated. In another embodiment, a 2-fold to 500-fold increase in gene expression, in another embodiment, a 2-fold to 100-fold increase in gene expression, in another embodiment, a 2-fold to 50-fold increase in gene expression, in another embodiment, a 2-fold to 20-fold increase in gene expression, in another embodiment, a 2-fold to 10-fold increase in gene expression, in another embodiment, a 3-fold to 5-fold increase in gene expression is contemplated.

In another embodiment, optimized gene expression may be an increase in gene expression under particular environmental conditions. In another embodiment, optimized gene expression may comprise a decrease in gene expression, which, in one embodiment, may be only under particular environmental conditions.

In another embodiment, optimized gene expression is an increased duration of gene expression. According to this aspect and in one embodiment, a 2-fold through 1000-fold increase in the duration of gene expression compared to wild-type is contemplated. In another embodiment, a 2-fold to 500-fold increase in the duration of gene expression, in another embodiment, a 2-fold to 100-fold increase in the duration of gene expression, in another embodiment, a 2-fold to 50-fold increase in the duration of gene expression, in another embodiment, a 2-fold to 20-fold increase in the duration of gene expression, in another embodiment, a 2-fold to 10-fold increase in the duration of gene expression, in another embodiment, a 3-fold to 5-fold increase in the duration of gene expression is contemplated. In another embodiment, the increased duration of gene expression is compared to gene expression in non-vector-expressing controls, or alternatively, compared to gene expression in wild-type-vector-expressing controls.

Expression in mammalian cells is hampered, in one embodiment, by transcriptional silencing, low mRNA half-life, alternative splicing events, premature polyadenylation, inefficient nuclear translocation and availability of rare tRNAs pools. The source of many problems in mammalian expressions is found within the message encoding the transgene including in the autologous expression of many crucial mammalian genes as well. The optimization of mammalian RNAs may include modification of cis acting elements, adaptation of its GC-content, modifying codon bias with respect to non-limiting tRNAs pools of the mammalian cell, avoiding internal homologous regions and excluding RNAi's.

Therefore, in one embodiment, when relying on carefully designed synthetic genes, stable messages with prolonged half-lives, constitutive nuclear export and high level protein production within the mammalian host can be expected.

Thus, in one embodiment, optimizing a gene entails adapting the codon usage to the codon bias of host genes, which in one embodiment, are Homo sapiens genes; adjusting regions of very high (>80%) or very low (<30%) GC content; avoiding one or more of the following cis-acting sequence motifs: internal TATA-boxes, chi-sites and ribosomal entry sites; AT-rich or GC-rich sequence stretches; ARE, INS, CRS sequence elements; repeat sequences and RNA secondary structures; (cryptic) splice donor and acceptor sites, branch points; or a combination thereof. In one embodiment, a gene is optimized for expression in homo sapien cells. In another embodiment, a gene is optimized for expression in micro-organs. In another embodiment, a gene is optimized for expression in dermal cells.

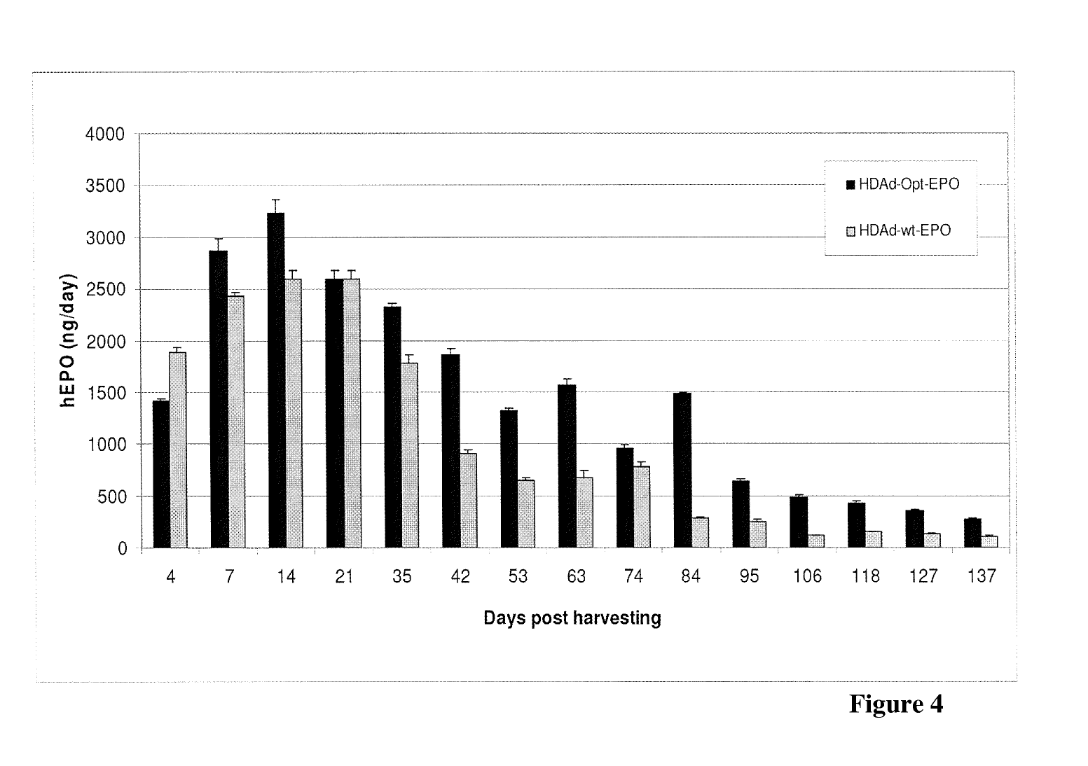

In one embodiment, as demonstrated herein, optimized genes, such as EPO, maintain an increase percent of peak expression levels for an extended period of time compared to both non-optimized EPO expressed from a gutless adenovirus vector or non-optimized EPO expressed from an adenovirus 5 vector (FIGS. 3 and 4).

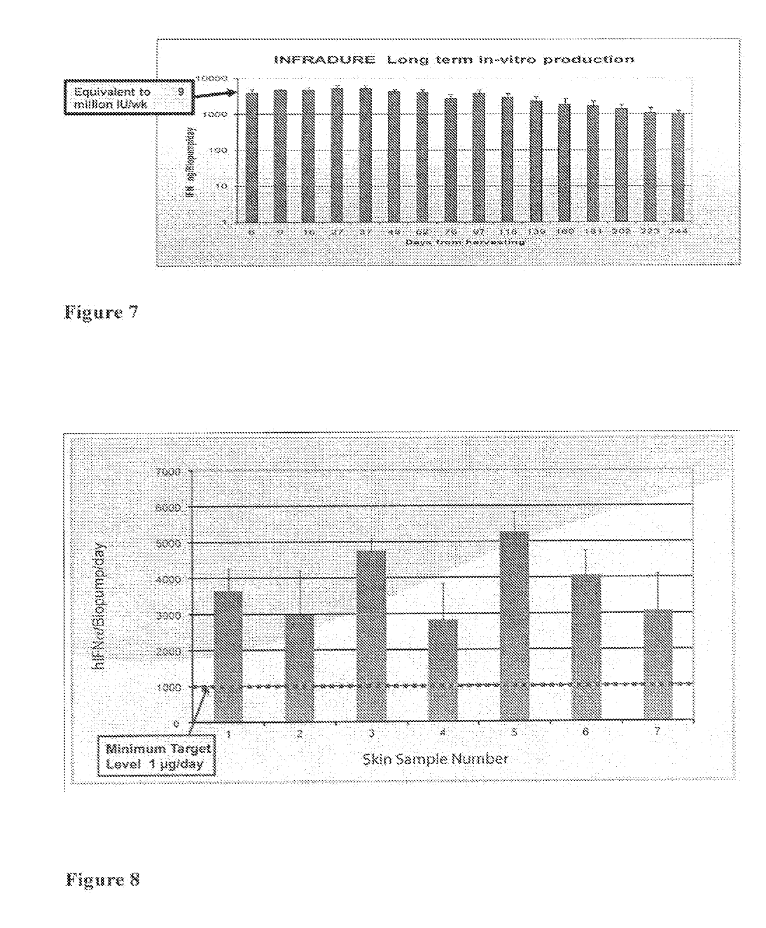

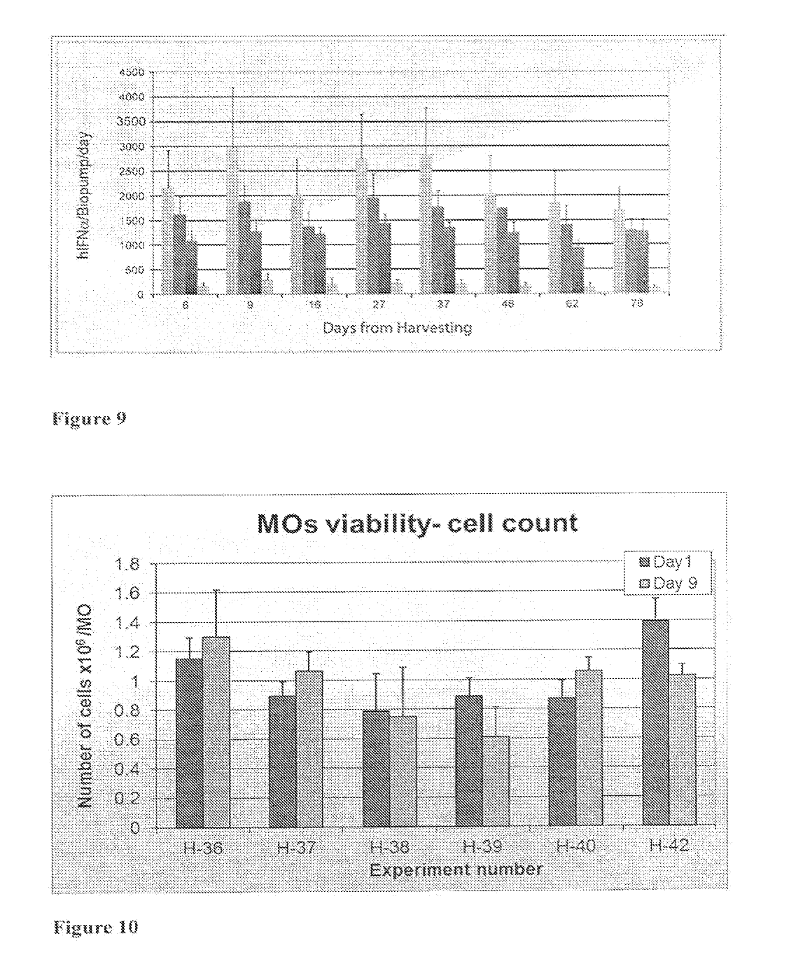

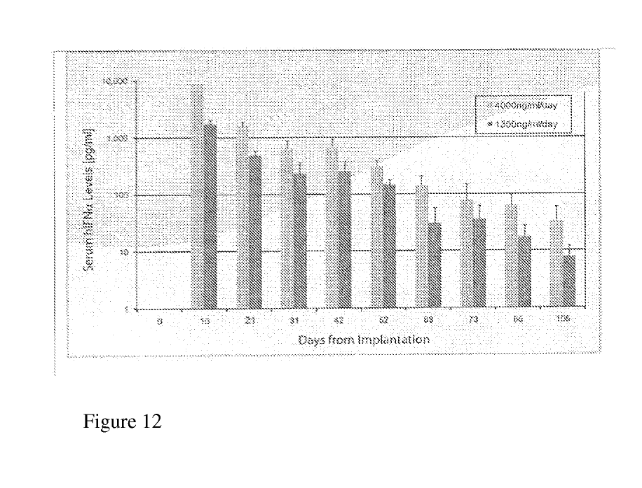

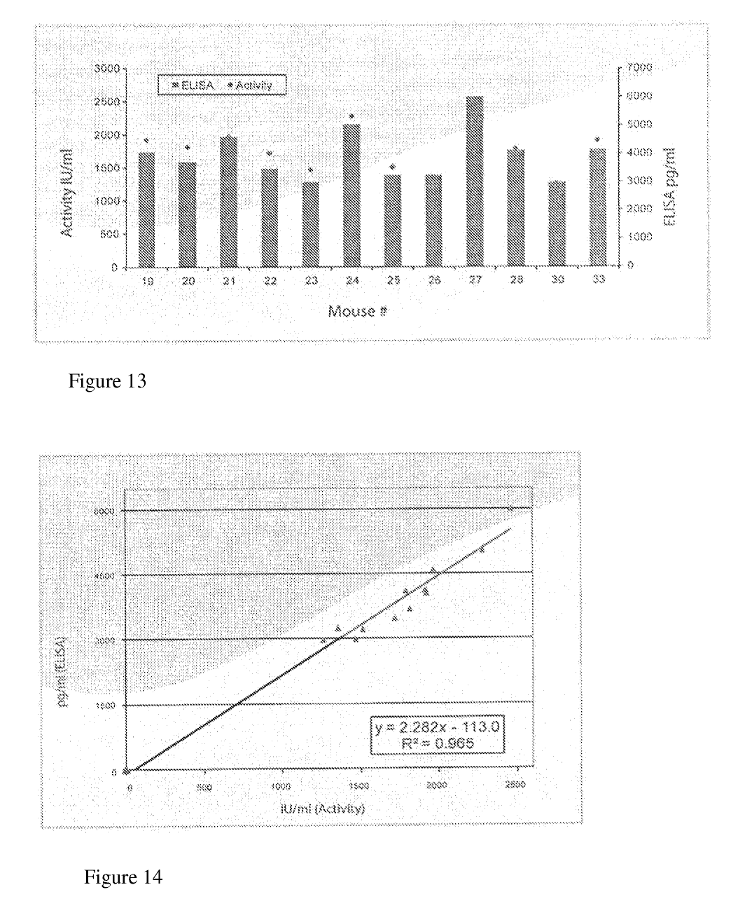

In one embodiment, as demonstrated herein, optimized genes, such as interferon (IFN), maintain an increased percent expression levels for an extended period of time in vitro (FIGS. 7 and 9). In another embodiment, as demonstrated herein, optimized genes, such as interferon (IFN), maintain an increased percent expression levels for an extended period of time in vivo (FIGS. 12 and 13).

In one embodiment, the term “gene” refers to a nucleic acid fragment that is capable of being expressed as a specific protein, including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

In one embodiment, the therapeutic nucleic acid may be any gene which encodes an RNA molecule (sense or antisense), peptide, polypeptide, glycoprotein, lipoprotein or combination thereof or to any other post modified polypeptide. In one embodiment of the invention, the gene of interest may be naturally expressed in the tissue sample. In another embodiment of this invention, the tissue sample may be genetically engineered so that at least one cell will express the gene of interest, which is either not naturally expressed by the cell or has an altered expression profile within the cell. In one embodiment, the therapeutic nucleic acid of the present invention may encode or the therapeutic polypeptide may be any of the proteins listed in United States Patent Application Publication Number US-2003-0152562-A1, which is incorporated herein by reference in its entirety.

In one embodiment, the genetically modified micro-organ is a genetically modified dermal micro-organ. “Dermal” micro-organs may comprise a plurality of dermis components, where in one embodiment; dermis is the portion of the skin located below the epidermis. These components may comprise skin fibroblast, epithelial cells, other cell types, bases of hair follicles, nerve endings, sweat and sebaceous glands, and blood and lymph vessels. In one embodiment, a dermal micro-organ may comprise fat tissue, wherein in another embodiment, a dermal micro-organ may not comprise fat tissue. Further details regarding dermal micro-organs, including methods of harvesting, maintaining in culture, and implanting said dermal micro-organs, are described in PCT Patent Application WO2004/099363, which is incorporated herein by reference in its entirety.

In another embodiment, the invention provides a method of providing a therapeutic polypeptide to a subject in need over a sustained period comprising providing one or more genetically modified micro-organs, said micro-organs comprising a vector comprising a nucleic acid sequence operably linked to one or more regulatory sequences; and implanting said genetically modified micro-organ in said subject, wherein said nucleic acid sequence encodes a therapeutic polypeptide and whereby the expression level of the therapeutic nucleic acid or polypeptide is increased by more than 5% over basal level and said increase is maintained for greater than one month. In another embodiment, the invention provides a method of providing a therapeutic polypeptide to a subject in need over a sustained period comprising providing one or more genetically modified micro-organs, said micro-organs comprising a vector comprising a nucleic acid sequence operably linked to one or more regulatory sequences; and implanting said genetically modified micro-organ in said subject, wherein said nucleic acid sequence encodes a therapeutic polypeptide and wherein said vector is a helper-dependent adenovirus vector. In another embodiment, the invention provides a method of providing a therapeutic polypeptide to a subject in need over a sustained period comprising providing one or more genetically modified micro-organs, said micro-organs comprising a vector comprising a nucleic acid sequence operably linked to one or more regulatory sequences; and implanting said genetically modified micro-organ in said subject, wherein said nucleic acid sequence encodes a therapeutic polypeptide and wherein said vector is a helper-dependent adenovirus vector.

In another embodiment, the methods described hereinabove provide a therapeutic nucleic acid to a subject in need wherein the expression level of the therapeutic nucleic acid or polypeptide is increased by more than 5% over basal level and said increase is maintained for greater than one hour, 3 hours, 6 hours, 9 hours, 12 hours, 18 hours, 1 day, or 2 days, wherein said vector is a helper-dependent adenovirus vector, or a combination thereof.

In one embodiment, this invention provides a therapeutic formulation as described hereinabove in which the therapeutic polypeptide is erythropoietin or wherein the therapeutic nucleic acid encodes erythropoietin. In another embodiment, this invention provides a long-lasting erythropoietin formulation comprising a genetically modified micro-organ, said micro-organ comprising a vector comprising a nucleic acid sequence operably linked to one or more regulatory sequences, wherein said nucleic acid sequence encodes erythropoietin and whereby said formulation increases erythropoietin levels by more than 5% over basal levels and said increased erythropoietin levels persist for greater than one month. In another embodiment, the invention provides a method of providing a therapeutic formulation to a subject in need in which the therapeutic polypeptide is erythropoietin or wherein the therapeutic nucleic acid encodes erythropoietin. In another embodiment, the invention provides a method of providing erythropoietin to a subject in need.

In another embodiment, this invention provides a method of delivering erythropoietin to a subject in need over a sustained period comprising: providing one or more genetically modified micro-organs, said micro-organs comprising a vector comprising a nucleic acid sequence operably linked to one or more regulatory sequences; and implanting said genetically modified micro-organ in said subject, wherein said nucleic acid sequence encodes erythropoietin and whereby erythropoietin levels are increased by more than 5% over basal levels and said increased erythropoietin levels persist for greater than one month.

In another embodiment, this invention provides a method of inducing formation of new blood cells in a subject in need over a sustained period comprising: providing one or more genetically modified micro-organs, said micro-organs comprising a vector comprising a nucleic acid sequence operably linked to one or more regulatory sequences; and implanting said genetically modified micro-organ in said subject, wherein said nucleic acid sequence encodes erythropoietin and whereby erythropoietin levels are increased by more than 5% over basal levels and said increased erythropoietin levels persist for greater than one month.

In one embodiment, erythropoietin (EPO) is a glycoprotein hormone involved in the maturation of erythroid progenitor cells into erythrocytes. In one embodiment, erythropoietin is essential in regulating levels of red blood cells in circulation. Naturally occurring erythropoietin is produced by the kidneys and liver, circulates in the blood, and stimulates the production of red blood cells in bone marrow, in one embodiment, in response to hypoxia.

In one embodiment, EPO of the compositions and methods of the instant invention may comprise glycosylation patterns similar to those of EPO extracted from human or animal urine, or in another embodiment, plasma.

The identification, cloning, and expression of genes encoding erythropoietin are described in U.S. Pat. Nos. 5,756,349; 5,955,422; 5,618,698; 5,547,933; 5,621,080; 5,441,868; and 4,703,008, which are incorporated herein by reference. A description of the purification of recombinant erythropoietin from cell medium that supported the growth of mammalian cells containing recombinant erythropoietin plasmids for example, are included in U.S. Pat. No. 4,667,016 to Lai et al, which is incorporated herein by reference. Recombinant erythropoietin produced by genetic engineering techniques involving the expression of a protein product in vitro from a host cell transformed with the gene encoding erythropoietin has been used to treat anemia resulting from chronic renal failure. Currently, EPO is used in the treatment of anemia of renal failure, the anemia associated with HIV infection in zidovudine (AZT) treated patients, and anemia associated with cancer chemotherapy. Administration of rhu-EPO has become routine in the treatment of anemia secondary to renal insufficiency, where doses of 50-75 u/kg given three times per week are used to gradually restore hematocrit and eliminate transfusion dependency.

Many cell surface and secretory proteins produced by eukaryotic cells are modified with one or more oligosaccharide groups called glycosylation, which can dramatically affect protein stability, secretion, and subcellular localization as well as biological activity. In one embodiment, both human urinary derived erythropoietin and recombinant erythropoietin (expressed in mammalian cells) having the amino acid sequence 1-165 of human erythropoietin comprise three N-linked and one O-linked oligosaccharide chains which together comprise about 40% of the total molecular weight of the glycoprotein. In one embodiment, non-glycosylated erythropoietin has greatly reduced in vivo activity compared to the glycosylated form but does retain some in vitro activity. In one embodiment, the EPO of the compositions and for use in the methods of the present invention are fully glycosylated, while in another embodiment, they are comprise some glycosylated residues, while in another embodiment, they are not glycosylated.

In one embodiment, the EPO gene may be a wild-type EPO gene, while in another embodiment, the EPO gene may be modified. In one embodiment, the modified EPO gene may be optimized.

In one embodiment, the EPO gene has a nucleic acid sequence that corresponds to that set forth in Genbank Accession Nos: X02158; AF202312; AF202311; AF202309; AF202310; AF053356; AF202306; AF202307; or AF202308 or encodes a protein sequence that corresponds to that set forth in Genbank Accession Nos: CAA26095; AAF23134; AAF17572; AAF23133; AAC78791; or AAF23132. In another embodiment, the EPO precursor gene has a nucleic acid sequence that corresponds to that set forth in Genbank Accession Nos: NM_{—}000799; M11319; BC093628; or BC111937 or encodes a protein sequence that corresponds to that set forth in Genbank Accession Nos: NP_{—}000790; AAA52400; AAH93628; or AAI11938.

In one embodiment, the formulations of the present invention may be used to treat a subject having anemia. In one embodiment, anemia is defined as “a pathologic deficiency in the amount of oxygen-carrying hemoglobin in the red blood cells.” Symptoms of anemia include fatigue, diminished ability to perform daily functions, impaired cognitive function, headache, dizziness, chest pain and shortness of breath, nausea, depression, pain, or a combination thereof. In one embodiment, anemia is associated with a poorer prognosis and increased mortality.

Anemia is often a consequence of renal failure due to decreased production of erythropoietin from the kidney. In another embodiment, anemia is caused by lowered red blood cell (erythroid) production by bone marrow due to cancer infiltration, lymphoma or leukemia, or marrow replacement. Other causes of anemia comprise, blood loss due to excessive bleeding such as hemorrhages or abnormal menstrual bleeding; cancer therapies, such as surgery, radiotherapy, chemotherapy, immunotherapy, or a combination thereof; infiltration or replacement of cancerous bone marrow; increased hemolysis, which in one embodiment is breakdown or destruction of red blood cells; low levels of erythropoietin, or a combination thereof. In one embodiment, anemia refers to Fanconi anemia, which in one embodiment, is an inherited anemia that leads to bone marrow failure (aplastic anemia) and often to acute myelogenous leukemia (AML). In another embodiment, anemia refers to Diamond Blackfan anemia, normocytic anemia, aplastic anemia, iron-deficiency anemia, vitamin deficiency anemia, Sideroblastic Anemia, Paroxysmal Nocturnal Hemoglobinuria, Anemia of Chronic Disease, Anemia in Kidney Disease and Dialysis, or a combination thereof. In another embodiment, the long-lasting EPO formulation of the instant invention is used for treating a diabetic subject. According to this aspect and in one embodiment, the EPO formulation of the instant invention may be used in conjunction with other treatments for diabetes known in the Art, including, inter alia, insulin administration, oral hypoglycemic drugs, which in one embodiment are sulfonurea drugs, which in one embodiment including inter alia glucotrol, glyburide, glynase and amaryl; glucophage, thiazolidinediones including inter alia rezulin, actos and avandia; or a combination thereof. In another embodiment, the long-lasting EPO formulation of the instant invention is used for treating a subject suffering from chronic kidney disease, while in another embodiment, is used for treating a subject suffering from end-stage renal disease. In another embodiment, the formulations of the instant invention are used for subjects that are susceptible to the above-mentioned diseases or conditions.

It is to be understood that the formulations and methods of this invention may be used to treat anemia, regardless of the cause of anemia and whether or not the cause of anemia is known.

In one embodiment, the formulations and method of the present invention may be administered with other treatments that are effective in treating anemia. In one embodiment, other treatments include iron supplements, vitamin B12 supplements, additional sources of erythropoietin, androgens, growth factors such as G-CSF, or a combination thereof. In another embodiment, the formulations and method of the present invention may be administered in conjunction with other treatments such as blood and marrow stem cell transplants.

In one embodiment, this invention provides a therapeutic formulation as described hereinabove in which the therapeutic polypeptide is interferon or in which the therapeutic nucleic acid encodes interferon, which in one embodiment, is interferon alpha, which in one embodiment, is interferon alpha 2a. In another embodiment, the present invention provides a long-lasting interferon-alpha formulation comprising a genetically modified micro-organ, said micro-organ comprising a vector comprising a nucleic acid sequence operably linked to one or more regulatory sequences, wherein said nucleic acid sequence encodes interferon-alpha and whereby said formulation increases interferon-alpha levels by more than 5% over basal levels and said increased interferon-alpha levels persist for greater than one month. In another embodiment, the invention provides a method of providing a therapeutic formulation to a subject in need in which the therapeutic polypeptide is interferon, or in which the therapeutic nucleic acid encodes, interferon, which in one embodiment, is interferon alpha, which in one embodiment, is interferon alpha 2a. In another embodiment, the invention provides a method of providing a therapeutic polypeptide which is interferon, which in one embodiment, is interferon alpha, which in one embodiment, is interferon alpha 2a to a subject in need.

In one embodiment, interferons are multi-functional cytokines that are capable of producing pleitrophic effects on cells, such as anti-viral, anti-proliferative and anti-inflammatory effects. Because of these cellular responses to interferons, interferon-alpha and interferon-beta have been found to be clinically useful in the treatment of viral, proliferative and inflammatory diseases such as multiple sclerosis, hepatitis B, hepatitis C, hepatitis D including chronic hepatitis D and several forms of cancer. Interferon therapies may also have potential use for the treatment of other inflammatory diseases, viral diseases and proliferative diseases. Thus, a subject in need of interferons may have one or all of the above-mentioned diseases or conditions.

In one embodiment, the formulations of the present invention may be used to treat a subject having hepatitis. In one embodiment, the formulations may be used to treat a subject having hepatitis B. In another embodiment, the formulations may be used to treat a subject having hepatitis C. In yet another embodiment, the formulations may be used to treat a subject having hepatitis D. In still another embodiment, the formulations may be used to treat a subject having a combination of hepatitis B and hepatitis D. In one embodiment, the subject having hepatitis C, has hepatitis C, genotype 1. In another embodiment, the subject has hepatitis C, genotype 2. In yet another embodiment, the subject has hepatitis C, genotype 3. In still another embodiment, the subject has hepatitis C, genotype 4, 5, 6, 7, 8, 9, 10 or 11. In a further embodiment, the subject has hepatitis C, with a combination of genotypes.

In another embodiment, the hepatitis C infection is a hepatitis C genotype 5 infection. In one embodiment, a method of this invention if for treating hepatitis in a human subject in need over a sustained period of time comprising the steps of: (a) providing at least one genetically modified micro-organ that expresses and secretes interferon, said genetically modified micro-organ comprising a helper dependent adenovirus vector comprising a nucleic acid sequence encoding interferon operably linked to one or more regulatory sequences; (b) determining interferon secretion levels of said at least on genetically modified micro-organ in vitro; (c) implanting said at least one genetically modified micro-organ in said human subject at an effective dosage; and either (d) measuring interferon in the serum of said human subject, and/or (e) measuring levels of hepatic virus in said human subject. In one embodiment, the interferon is selected from the group including interferon alpha, interferon beta and interferon gamma. In one embodiment, the interferon alpha is interferon alpha 2a or interferon alpha 2b.

In one embodiment, the methods of this invention comprising use of a nucleic acid sequence encoding said interferon is optimized for increased expression levels, increased duration of expression, or a combination thereof. In one embodiment the methods comprise use of an optimized nucleic acid sequence that is greater than 85% homologous to SEQ ID No: 1.

In one embodiment, the methods of this invention treat hepatitis B. In another embodiment, the methods treat hepatitis C. In yet another embodiment, the methods treat hepatitis C, genotype 1. In still another embodiment, the methods treat hepatitis C, genotype 2. In a further embodiment, the methods treat hepatitis C, genotype 3. In one embodiment, the methods of this invention treat hepatitis that is hepatitis C caused by a combination of genotypes. In another embodiment, the methods of this invention treat hepatitis D. In still another embodiment, the methods of this invention treat chronic hepatitis D.

As used herein, “hepatitis B” refers to an irritation and swelling (inflammation) of the liver due to infection with the hepatitis B virus (HBV). Hepatitis B infection can be spread through having contact with the blood, semen, vaginal fluids, and other body fluids of someone who already has a hepatitis B infection. Infection can be spread through: blood transfusions (not common in the United States); direct contact with blood in health care settings; sexual contact with an infected person; tattoo or acupuncture with unclean needles or instruments; shared needles during drug use; shared personal items (such as toothbrushes, razors, and nail clippers) with an infected person; and the hepatitis B virus can be passed to an infant during childbirth if the mother is infected.

As used herein, “hepatitis C” refers to an infectious disease affecting primarily the liver, caused by the hepatitis C virus (HCV). The infection is often asymptomatic, but chronic infection can lead to scarring of the liver and ultimately to cirrhosis, which is generally apparent after many years. In some cases, those with cirrhosis will go on to develop liver failure, liver cancer or life-threatening esophageal and gastric varices. HCV is spread primarily by blood-to-blood contact associated with intravenous drug use, poorly sterilized medical equipment and transfusions.

As used herein hepatitis D refers to an inflammation of the liver caused by the hepatitis D virus (HDV). HDV is a defective RNA virus that cannot replicate autonomously but can assemble as a virion only if provided with a lipoprotein envelop by the hepatitis B virus (HBV). Therefore, transmission of HDV can occur only via simultaneous infection with HBV (co-infection) or via superimposition on chronic hepatitis B or hepatitis B carrier state (super-infection). Whereas co-infection generally leads to a self-limiting acute hepatitis, super-infection causes a severe acute hepatitis that in 80-90% of infected people progresses to chronicity (chronic hepatitis D).

The chronic hepatitis caused by HDV superinfection differs from the one caused by HBV monoinfection. Chronic hepatitis D is significantly more likely than chronic hepatitis B to progress to more severe disease states and death. This notion is supported by several studies that have documented increased occurrence of elevated levels of aminotransferase and bilirubin, and symptomatic hepatitis and jaundice, as well as more rapid progression to cirrhosis and possibly increased risk of developing hepatocellular carcinoma. The distinctiveness of chronic hepatitis D and chronic hepatitis B is underscored by the finding that HDV frequently suppresses HBV replication, making HDV replication rather than HBV replication a major determinant of disease progression in chronic hepatitis D. The two diseases are also distinct with regard to therapeutic approaches for their treatment. Whereas there are multiple approved therapies for hepatitis B, including interferon and nucleoside/nucleotide analogs, none are approved for hepatitis D. For example, nucleosides/nucleotides have shown no efficacy in hepatitis D, and there are limited, albeit positive, data on the effectiveness of interferon in treating hepatitis D.

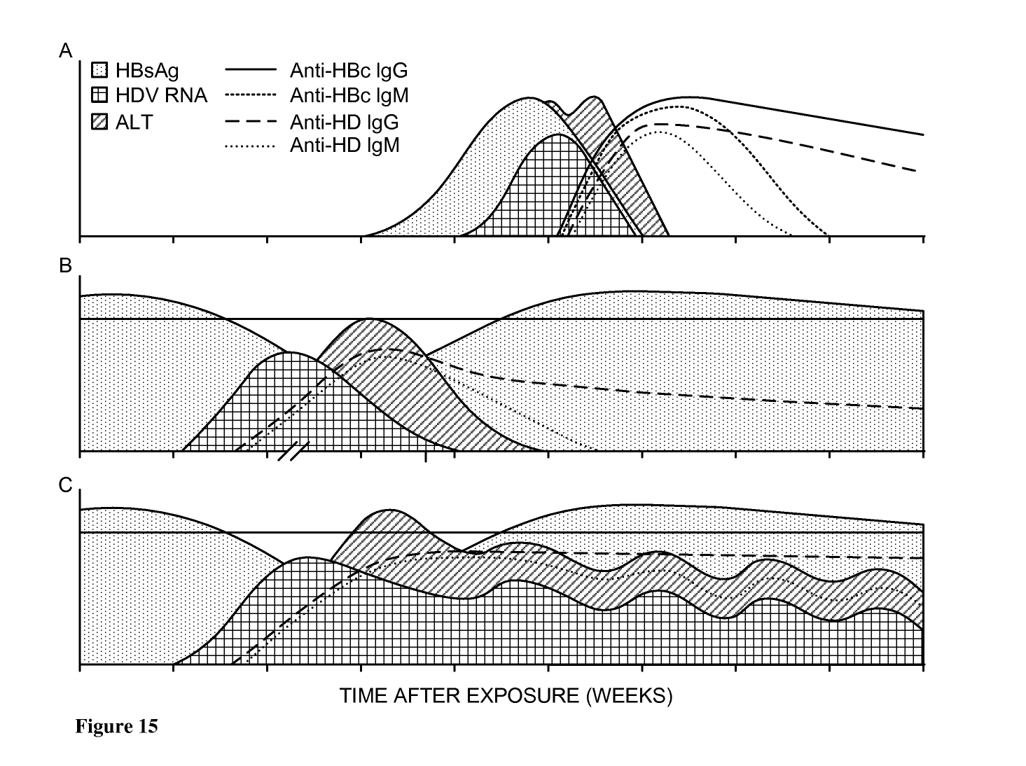

Patients with hepatitis D may be identified using serologic test, for example see FIG. 15.

As used herein, “treatment” or “treating” of hepatitis refers to reducing the hepatitis virus, for example, reducing hepatitis B virus or reducing hepatitis C virus or reducing hepatitis D virus or reducing a combination of these viruses thereof. In one embodiment, treatment results in reduction of viral load, e.g., Hepatitis C RNA reduction or Hepatitis B DNA reduction. Reduction of virus number or viral load may be assessed by testing for loss of viral DNA, e.g., loss of Hepatitis B viral DNA. Alternatively, loss of virus number or viral load may be assessed by loss of specific viral antigens such as Hepatitis B “e” antigen (HBeAg) or Hepatitis B “surface” antigen (HBsAg).

In one embodiment a Biopump of this invention expressing and secreting interferon could fulfill an unmet need for reliable interferon therapy for hepatitis D, a particularly aggressive form of hepatitis for which years of interferon therapy is the only effective treatment. Though an orphan disease in the U.S., hepatitis D is becoming a significant cause of death in Europe.

In one embodiment, a Biopump of this invention expressing and secreting interferon may address the unmet need in the treatment of hepatitis B, namely to eliminate the hepatitis B virus (HBV), not just contain it. In one embodiment, treatment of hepatitis using a method of this invention causes the loss of HBV DNA, HBeAg, HBsAg, or any combination of these.

In one embodiment, a method of this invention treating hepatitis eliminates the hepatitis B surface antigen (HBsAg). In certain instances, following loss of the HBsAg, there is seroconversion in which the body produces antibodies against the HBsAg. Sero-conversion is attained in only a small percentage of patients using oral antiviral agents and only after long-term use. Surface antigen loss and sero-conversion against HBsAg has been reported to be improved by one-to-two years of interferon alpha therapy. However, today this requires the patient to endure weekly injections of pegylated interferon alpha with its associated side effects, creating a significant challenge in patient compliance to complete treatment. A Biopump of this invention expressing and secreting interferon alpha has the potential to provide a much more practical and patient-compliant way to attain sero-conversion or surface antigen loss in a larger proportion of patients by having the patient's own tissue produce and deliver the protein instead of using injections, whether supplemental to oral nucleotide/nucleoside treatments, or on its own.

There are three major classes of interferons: alpha (α), beta (β), and gamma (γ). Aside from their antiviral and anti-oncogenic properties, interferons activate macrophage and natural killer lymphocyte, and enhance major histocompatibility complex glycoprotein classes I and II. Interferon-α is secreted by leukocytes (B-cells and T-cells). Interferon-β is secreted by fibroblasts, and interferon-γ is secreted by T-cells and natural killer lymphocytes.

In one embodiment, the therapeutic polypeptide is interferon alpha, in another embodiment, interferon beta, or in another embodiment, interferon gamma. In another embodiment, the therapeutic polypeptide is any subtype of interferon alpha, including but not limited to: 1, 2, 4, 5, 6, 7, 8, 10, 13, 14, 16, 17, or 21. In another embodiment, the therapeutic polypeptide is interferon omega, epsilon, kappa, or a homolog thereof. In another embodiment, the therapeutic polypeptide is interferon lambda or a homolog thereof. In another embodiment, the therapeutic polypeptide is any subtype of interferon lambda including but not limited to: Interleukin (IL) 28A, IL28B, or IL29. In another embodiment, the therapeutic polypeptide is interferon zeta, nu, tau, delta, or a homolog thereof.

In one embodiment, IFNs bind to a specific cell surface receptor complex, which in one embodiment is interferon alpha receptor (IFNAR) comprising IFNAR1 and IFNAR2 chains, in another embodiment is interferon gamma receptor (IFNGR) complex, which comprises two IFNGR1 and two IFNGR2 subunits, in another embodiment is a receptor complex comprising IL10R2 and IFNLR1. In one embodiment, interferons signal through the JAK-STAT signaling pathway.

In one embodiment, the interferon of the formulations and methods of the instant invention are interferon alpha. In another embodiment, the interferon of the formulations and methods of the instant invention are interferon alpha2b. In one embodiment, IFN-alpha-2b is a recombinant, non-glycosylated 165-amino acid alpha interferon protein comprising the gene for IFN-alpha-2b from human leukocytes. IFN-alpha-2b is a type I, water-soluble interferon with a molecular weight of 19,271 daltons (19.271 kDa). In one embodiment, IFN-alpha-2b has a specific activity of about 2.6×108 (260 million) International Units/mg as measured by HPLC assay.

In one embodiment, IFN-alpha-2b is one of the Type I interferons, which belong to the larger helical cytokine superfamily, which includes growth hormones, interleukins, several colony-stimulating factors and several other regulatory molecules. All function as regulators of cellular activity by interacting with cell-surface receptor complexes, known as IFNAR1 and IFNAR2, and activating various signaling pathways. Interferons produce antiviral and anti-proliferative responses in cells.

In one embodiment, a long-lasting IFN-alpha formulation of the present invention may be used for the prevention or treatment of hairy cell leukemia, venereal warts, Kaposi's Sarcoma, chronic non-A, non-B hepatitis, hepatitis B, or a combination thereof. In another embodiment, a long-lasting IFN-alpha formulation of the present invention may be administered to a subject that is susceptible to one of the above-mentioned diseases or conditions or has been or will be exposed to an infectious agent, as described herein. In another embodiment, a long-lasting IFN-alpha formulation invention may be used for the prevention or treatment of hepatitis C. In one embodiment, the hepatitis C is genotype 1. In another embodiment, the hepatic C is genotype 2. In yet another embodiment, the hepatitis C is genotype 3. In still another embodiment, the hepatitis C is caused by a combination of genotypes. According to this aspect and in one embodiment, the formulations of the present invention may be administered concurrently or alternately with other hepatitis C treatments, including inter alia, ribavarin, interferons, pegylated interferons or a combination thereof.

In another embodiment, a long-lasting IFN-alpha formulation may be used or evaluated alone or in conjunction with chemotherapeutic agents in a variety of other cellular proliferation disorders, including chronic myelogenous leukemia, multiple myeloma, superficial bladder cancer, skin cancers (including, inter alia, basal cell carcinoma and malignant melanoma), renal cell carcinoma, ovarian cancer, low grade lymphocytic and cutaneous T cell lymphoma, and glioma. In another embodiment, a long-lasting IFN-alpha formulation may be used for the prevention or treatment of solid tumors that arise from lung, colorectal and breast cancer, alone or with other chemotherapeutic agents. In another embodiment, a long-lasting IFN-alpha formulation may be used for the prevention or treatment of multiple sclerosis. In another embodiment, a long-lasting IFN-alpha formulation may be used for the prevention or treatment of histiocytic diseases, which in one embodiment is Erdheim-Chester disease (ECD), which in one embodiment is a potentially fatal disorder that attacks the body's connective tissue and in one embodiment is caused by the overproduction of histiocytes, which in one embodiment, accumulate in loose connective tissue, causing it to become thickened and dense. In another embodiment, a long-lasting IFN-alpha formulation may be used for the prevention or treatment of severe ocular Behcet's disease.

In one embodiment, the interferon alpha gene has a nucleic acid sequence that corresponds to that set forth in Genbank Accession Nos: K01900; M11003; or M71246, or encodes a protein sequence that corresponds to that set forth in Genbank Accession Nos: AAA52716; AAA52724; or AAA52713. In one embodiment, the interferon beta gene has a nucleic acid sequence that corresponds to that set forth in Genbank Accession Nos: M25460; AL390882; or CH236948, or encodes a protein sequence that corresponds to that set forth in Genbank Accession Nos: AAC41702; CAH70160; or EAL24265. In one embodiment, the interferon gamma gene has a nucleic acid sequence that corresponds to that set forth in Genbank Accession Nos: J00219; AF506749; NM_{—}000619; or X62468, or encodes a protein sequence that corresponds to that set forth in Genbank Accession Nos: AAB59534; AAM28885; NP_{—}000610; or CAA44325. In another embodiment, the interferon alpha gene has a nucleic acid sequence as presented in SEQ ID No: 1, while in another embodiment, the interferon alpha gene has an amino acid sequence as presented in SEQ ID No: 5. In another embodiment, the interferon alpha gene has a nucleic acid that is homologous to that presented in SEQ ID No: 4.

In another embodiment, the present invention provides a method of delivering interferon-alpha to a subject in need over a sustained period comprising: providing one or more genetically modified micro-organs, said micro-organs comprising a vector comprising a nucleic acid sequence operably linked to one or more regulatory sequences; and implanting said genetically modified micro-organ in said subject, wherein said nucleic acid sequence encodes interferon-alpha and whereby interferon-alpha levels are increased by more than 5% over basal levels and said increased interferon-alpha levels persist for greater than one month.

In one embodiment, the formulations and methods of the present invention provide a nucleic acid optimized for increased expression levels, duration, or a combination thereof of a therapeutic polypeptide encoded by said nucleic acid. In another embodiment, the invention provides a nucleic acid sequence with greater than 85% homology to SEQ ID No: 1, a vector comprising such a nucleic acid sequence, and a cell comprising such as vector.

In another embodiment, the invention provides a nucleic acid sequence with greater than 85% homology to SEQ ID No: 4, a vector comprising such a nucleic acid sequence, and a cell comprising such as vector.

The term “homology”, as used herein, when in reference to any nucleic acid sequence indicates a percentage of nucleotides in a candidate sequence that is identical with the nucleotides of a corresponding native nucleic acid sequence.

In one embodiment, the terms “homology”, “homologue” or “homologous”, in any instance, indicate that the sequence referred to, exhibits, in one embodiment at least 70% correspondence with the indicated sequence. In another embodiment, the nucleic acid sequence exhibits at least 72% correspondence with the indicated sequence. In another embodiment, the nucleic acid sequence exhibits at least 75% correspondence with the indicated sequence. In another embodiment, the nucleic acid sequence exhibits at least 77% correspondence with the indicated sequence. In another embodiment, the nucleic acid sequence exhibits at least 80% correspondence with the indicated sequence. In another embodiment, the nucleic acid sequence exhibits at least 82% correspondence with the indicated sequence. In another embodiment, the nucleic acid sequence exhibits at least 85% correspondence with the indicated sequence. In another embodiment, the nucleic acid sequence exhibits at least 87% correspondence with the indicated sequence. In another embodiment, the nucleic acid sequence exhibits at least 90% correspondence with the indicated sequence. In another embodiment, the nucleic acid sequence exhibits at least 92% correspondence with the indicated sequence. In another embodiment, the nucleic acid sequence exhibits at least 95% or more correspondence with the indicated sequence. In another embodiment, the nucleic acid sequence exhibits 95%-100% correspondence to the indicated sequence. Similarly, reference to a correspondence to a particular sequence includes both direct correspondence, as well as homology to that sequence as herein defined.

Homology may be determined by computer algorithm for sequence alignment, by methods well described in the art. For example, computer algorithm analysis of nucleic acid sequence homology may include the utilization of any number of software packages available, such as, for example, the BLAST, DOMAIN, BEAUTY (BLAST Enhanced Alignment Utility), GENPEPT and TREMBL packages.

An additional means of determining homology is via determination of nucleic acid sequence hybridization, methods of which are well described in the art (See, for example, “Nucleic Acid Hybridization” Hames, B. D., and Higgins S. J., Eds. (1985); Sambrook et al., 1989, Molecular Cloning, A Laboratory Manual, (Volumes 1-3) Cold Spring Harbor Press, N.Y.; and Ausubel et al., 1989, Current Protocols in Molecular Biology, Green Publishing Associates and Wiley Interscience, N.Y). In one embodiment, methods of hybridization may be carried out under moderate to stringent conditions. Hybridization conditions being, for example, overnight incubation at 42° C. in a solution comprising: 10-20% formamide, 5×SSC (150 mM NaCl, 15 mM trisodium citrate), 50 mM sodium phosphate (pH 7.6), 5×Denhardt's solution, 10% dextran sulfate, and 20 μg/ml denatured, sheared salmon sperm DNA.

In one embodiment, the present invention provides therapeutic formulations comprising micro-organs and methods of use thereof. In one embodiment, the preparation of therapeutic micro-organs comprises (a) obtaining a plurality of micro-organ explants from a donor subject, each of the plurality of micro-organ explants comprises a population of cells, each of the plurality of micro-organ explants maintaining a microarchitecture of an organ from which it is derived and at the same time having dimensions selected so as to allow diffusion of adequate nutrients and gases to cells in the micro-organ explants and diffusion of cellular waste out of the micro-organ explants so as to minimize cellular toxicity and concomitant death due to insufficient nutrition and accumulation of the waste in the micro-organ explants; (b) genetically modifying the plurality of micro-organ explants, so as to obtain a plurality of genetically modified micro-organ explants, said micro-organs comprising and secreting the proteins differing by the at least one amino acid; and (c) implanting the plurality of genetically modified micro-organ explants within a plurality of recipient subjects.

In one embodiment, the preparation of therapeutic micro-organs is performed as described in PCT patents WO 03/006669, WO 03/035851, and WO 04/099363, which are incorporated herein by reference in their entirety.

Methods for the preparation and processing of micro-organs into genetically modified micro-organs are disclosed in WO2004/099363, incorporated herein by reference in their entirety. Micro-organs comprise tissue dimensions defined such that diffusion of nutrients and gases into every cell in the three dimensional micro-organ, and sufficient diffusion of cellular wastes out of the explant, is assured. Ex vivo maintenance of the micro-organs, which in one embodiment, is in minimal media, can continue for an extended period of time, whereupon controlled ex vivo transduction incorporating desired gene candidates within cells of the micro-organs using viral or non-viral vectors occurs, thus creating genetically modified micro-organs.

In one embodiment, micro-organs are harvested using a drill and coring needle, as described hereinbelow. In another embodiment, micro-organs are harvested using a harvesting system that utilizes a vacuum to hold the skin taut and open the slits during insertion of the coring drill. In another embodiment, any tool which may be used to harvest dermal tissue may be used to harvest micro-organs of the appropriate size, including but not limited to those tools and methods described in PCT Application WO 04/099363.

Incorporation of recombinant nucleic acid within the micro-organs to generate genetically modified micro-organs or biopumps can be accomplished through a number of methods well known in the art. Nucleic acid constructs can be utilized to stably or transiently transduce the micro-organ cells. In stable transduction, the nucleic acid molecule is integrated into the micro-organ cells genome and as such it represents a stable and inherited trait. In transient transduction, the nucleic acid molecule is maintained in the transduced cells as an episome and is expressed by the cells but it is not integrated into the genome. Such an episome can lead to transient expression when the transduced cells are rapidly dividing cells due to loss of the episome or to long term expression wherein the transduced cells are non-dividing cells.

Typically the nucleic acid sequence is subcloned within a particular vector, depending upon the preferred method of introduction of the sequence to within the micro-organs, as described hereinabove. Once the desired nucleic acid segment is subcloned into a particular vector it thereby becomes a recombinant vector.

In one embodiment, micro-organs are incubated at 32° C. before and after genetic modification, while in another embodiment, they are incubated at 37° C. In another embodiment, micro-organs are incubated at 33° C., 34° C., 35° C., 36° C., 38° C., 39° C., 40° C., 28° C., 30° C., 31° C., 25° C., 42° C., or 45° C.

In one embodiment, micro-organs are incubated at 10% CO₂ before and after genetic modification, while in another embodiment, they are incubated at 5% CO₂. In another embodiment, micro-organs are incubated at 12% CO₂, 15% CO₂, 17% CO₂, or 20% CO₂. In another embodiment, micro-organs are incubated at 2% CO₂, 6% CO₂, 7% CO₂, 8% CO₂, or 9% CO₂.

In another embodiment, incubation temperatures, CO₂ concentrations, or a combination thereof may be kept at a single temperature or concentration before, during, and after genetic modification, while in another embodiment, incubation temperatures, CO₂ concentrations, or a combination thereof may be adjusted at different points before, during, and after genetic modification of micro-organs.

In another embodiment, micro-organs are incubated at 85-100% humidity, which in one embodiment is 95% humidity, in another embodiment, 90% humidity, and in another embodiment, 98% humidity.

In one embodiment, the levels of therapeutic nucleic acids or polypeptides may be detected using any method known in the art. The efficacy of a particular expression vector system and method of introducing nucleic acid into a cell can be assessed by standard approaches routinely used in the art. For example, DNA introduced into a cell can be detected by a filter hybridization technique (e.g., Southern blotting) and RNA produced by transcription of introduced DNA can be detected, for example, by Northern blotting, RNase protection or reverse transcriptase-polymerase chain reaction (RT-PCR). The gene product can be detected by an appropriate assay, for example by immunological detection of a produced protein, such as with a specific antibody, or by a functional assay to detect a functional activity of the gene product, such as an enzymatic assay. In one embodiment, ELISA, Western blots, or radioimmunoassay may be used to detect proteins. If the gene product of interest to be expressed by a cell is not readily assayable, an expression system can first be optimized using a reporter gene linked to the regulatory elements and vector to be used. The reporter gene encodes a gene product which is easily detectable and, thus, can be used to evaluate efficacy of the system. Standard reporter genes used in the art include genes encoding β-galactosidase, chloramphenicol acetyl transferase, luciferase and human growth hormone.

Thus, in one embodiment, therapeutic polypeptide or nucleic acid expression levels are measured in vitro, while in another embodiment, therapeutic polypeptide or nucleic acid expression levels are measured in vivo. In one embodiment, in vitro determination of polypeptide or nucleic acid expression levels, which in one embodiment, is EPO levels and in another embodiment, IFN-alpha levels, allows a determination of the number of micro organs to be implanted in a patient via determining the secretion level of a therapeutic agent by a micro-organ in vitro; estimating a relationship between in vitro production and secretions levels and in vivo serum levels of the therapeutic agent; and determining an amount of the therapeutic formulation to be implanted, based on the determined secretion level and the estimated relationship.

In another preferred embodiment of this invention, polynucleotide(s) can also include trans-, or cis-acting enhancer or suppresser elements which regulate either the transcription or translation of endogenous genes expressed within the cells of the micro-organs, or additional recombinant genes introduced into the micro-organs. Numerous examples of suitable translational or transcriptional regulatory elements, which can be utilized in mammalian cells, are known in the art.

For example, transcriptional regulatory elements comprise cis- or trans-acting elements, which are necessary for activation of transcription from specific promoters [(Carey et al., (1989), J. Mol. Biol. 209:423-432; Cress et al., (1991), Science 251:87-90; and Sadowski et al., (1988), Nature 335:5631-564)].

Translational activators are exemplified by the cauliflower mosaic virus translational activator (TAV) [see for example, Futterer and Hohn, (1991), EMBO J. 10:3887-3896]. In this system a bi-cistronic mRNA is produced. That is, two coding regions are transcribed in the same mRNA from the same promoter. In the absence of TAV, only the first cistron is translated by the ribosomes, however, in cells expressing TAV, both cistrons are translated.

The polynucleotide sequence of cis-acting regulatory elements can be introduced into cells of micro-organs via commonly practiced gene knock-in techniques. For a review of gene knock-in/out methodology see, for example, U.S. Pat. Nos. 5,487,992, 5,464,764, 5,387,742, 5,360,735, 5,347,075, 5,298,422, 5,288,846, 5,221,778, 5,175,385, 5,175,384, 5,175,383, 4,736,866 as well as Burke and Olson, Methods in Enzymology, 194:251-270, 1991; Capecchi, Science 244:1288-1292, 1989; Davies et al., Nucleic Acids Research, 20 (11) 2693-2698, 1992; Dickinson et al., Human Molecular Genetics, 2(8):1299-1302, 1993; Duff and Lincoln, “Insertion of a pathogenic mutation into a yeast artificial chromosome containing the human APP gene and expression in ES cells”, Research Advances in Alzheimer's Disease and Related Disorders, 1995; Huxley et al., Genomics, 9:742-750 1991; Jakobovits et al., Nature, 362:255-261 1993; Lamb et al., Nature Genetics, 5: 22-29, 1993; Pearson and Choi, Proc. Natl. Acad. Sci. USA, 1993, 90:10578-82; Rothstein, Methods in Enzymology, 194:281-301, 1991; Schedl et al., Nature, 362: 258-261, 1993; Strauss et al., Science, 259:1904-1907, 1993, WO 94/23049, WO 93/14200, WO 94/06908 and WO 94/28123 also provide information.

Down-regulation of endogenous sequences may also be desired, in order to assess production of the recombinant product exclusively. Toward this end, antisense RNA may be employed as a means of endogenous sequence inactivation. Exogenous polynucleotide(s) encoding sequences complementary to the endogenous mRNA sequences are transcribed within the cells of the micro-organ. Down regulation can also be effected via gene knock-out techniques, practices well known in the art (“Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988)).

Over expression of the recombinant product may be desired as well. Overexpression may be accomplished by providing a high copy number of one or more coding sequences in the respective vectors. These exogenous polynucleotide sequences can be placed under transcriptional control of a suitable promoter of a mammalian expression vectors to regulate their expression. In another embodiment, multiple copies of the same gene or of several related genes may be used as a means to increase polypeptide or nucleic acid expression. In one embodiment, expression is stabilized by DNA elements, which in one embodiment are matrix-associating regions (MARs) or scaffold-associating regions (SARs).

In one embodiment, an adenoviral vector is the vector of the compositions and for use in the methods of the present invention. In an embodiment in which an adenoviral vector is used as a vector, the helper-dependent adenovirus system may be used in one embodiment, to prepare therapeutic polypeptide or nucleic acid-expressing helper-dependent adenovirus vector for transforming micro-organs. In one embodiment, such a helper-dependent adenovirus system comprises a helper-dependent adenovirus, a helper virus, and a producer cell line is used in the preparation of the formulation of the present invention is as described in Palmer and Ng, 2003 Mol Ther 8:846 and in Palmer and Ng, 2004 Mol Ther 10:792, which are hereby incorporated herein by reference in their entirety.

In one embodiment, a helper cell line, designated 293, which was transformed from human embryonic kidney cells by Ad5 DNA fragments and constitutively expresses E1 proteins is used to generate and propagate replication deficient adenoviral vectors. In another embodiment, helper cell lines may be derived from human muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells.

In one embodiment, micro-organs are maintained ex vivo for a period of time, which may range from several hours to several months. In one embodiment, they are maintained for several days, and in another embodiment, for several weeks prior to implantation. Without being limited by theory, in one embodiment, said incubation allows cells to process and break down viral proteins, which in one embodiment are viral capsids, present as a result of viral vector transduction. In one embodiment, such a turnover of capsid proteins occurs within 2-3 days, so that, in one embodiment, little if any viral capsid proteins remain by the 10^{th} day ex vivo. In one embodiment, the breaking down of viral capsids further reduces the immunogenicity of the formulations of the instant invention and increases the expression levels and expression duration of the gene or genes of interest. In another embodiment, said incubation allows the early HD-Ad vector-induced innate immune responses to occur in vitro, which in one embodiment, will not persist beyond 24 hours in the absence of Adeno gene transcription. In another embodiment, the later adaptive responses that normally follow the administration of transcription-competent first-generation-Ad vectors, which are predominantly characterized in one embodiment, by lymphocyte infiltration and in another embodiment by induction of Ad-specific CTL's, are not be elicited by HD-Ad vectors.

In one embodiment, the ex vivo micro-organ is exposed to viral vector at a dosage of 1.6-3×10⁹ infectious particles (ip)/ml, 3-4×10¹² viral particles/ml, or 2×10¹¹ viral particles/ml. In another embodiment, ex vivo micro-organs are exposed to viral vector at a dosage of 1×10³ to 1×10¹² viral particles/ml, in another embodiment from 1×10³ to 1×10⁹, and in another embodiment, from 1×10⁶ to 1×10⁹ and in another embodiment, 1×10⁶ to 1×10¹² viral particles/ml. In one embodiment, the dosage of viral particles/g body weight of subject that are administered to a subject within a micro-organ is less than 1×10³, and in another embodiment, less than 1×10², and in another embodiment, less than 1×10¹ viral particles/g body weight of subject.

In one embodiment, growth factors are used to increase the number of cells in the micro-organs.

In one embodiment, in vitro expression can be assessed prior to implantation, enabling the possibility for in vitro to in vivo correlation studies of expressed recombinant proteins.

In some embodiments of the invention, the amounts of tissue sample including a genetically modified cell(s) to be implanted are determined from one or more of: corresponding amounts of the therapeutic agent of interest routinely administered to such subjects based on regulatory guidelines, specific clinical protocols or population statistics for similar subjects, corresponding amounts of the therapeutic agent such as protein of interest specifically to that same subject in the case that he/she has received it via injections or other routes previously, subject data such as weight, age, physical condition, clinical status, pharmacokinetic data from previous tissue sample which includes a genetically modified cell administration to other similar subjects, response to previous tissue sample which includes a genetically modified cell administration to that subject, or a combination thereof. Thus, in one embodiment, the level of expression of gene products by one or more micro-organs is determined in vitro, a relationship between in vitro and in vivo therapeutic polypeptide or nucleic acid expression levels is determined or estimated, and the number of micro-organs to be implanted in a particular patient is determined based on the calculated or estimated relationship. The dosage of the therapeutic agent may be adjusted as described previously (WO2004/099363).

In one embodiment, a micro-organ or a genetically modified micro-organ may be maintained in vitro for a proscribed period of time until they are needed for implantation into a host. In one embodiment, a micro-organ or a genetically modified micro-organ may be maintained or stored in culture for between 1-16 days, between 1-8 weeks, or for 1-4 months. In another embodiment, the therapeutic agent, left in the supernatant medium surrounding the tissue sample, can be isolated and injected or applied to the same or a different subject.

Alternatively or additionally, a genetically modified micro-organ can be cryogenically preserved by methods known in the art, for example, without limitation, gradual freezing (0° C., −20° C., −80° C., −196° C.) in DMEM containing 10% DMSO, immediately after being formed from the tissue sample or after genetic alteration.

In one embodiment, the formulation of the instant invention may be implanted in an organ or system that is affected by a disease or disorder to be treated or prevented by a method or route which results in localization of the micro-organ at a desired site. In another embodiment, the location of the implanted formulation may be distal from an organ or system that is affected by a disease or disorder. Thus, while in one embodiment, the recombinant protein is released locally, in another embodiment, the recombinant protein diffuses to the lymphatic system, which in one embodiment, may ultimately lead to systemic distribution of the recombinant protein. Thus, the present invention provides for the use of therapeutic formulations in various concentrations to treat a disease or disorder manifesting in any part of the subject in need.

In one embodiment, the formulations of the invention may be implanted a single time for acute treatment of temporary conditions, or may be implanted more than one time, especially in the case of progressive, recurrent, or degenerative disease. In one embodiment, one or more formulations of the invention may be administered simultaneously, or in another embodiment, they may be administered in a staggered fashion. In one embodiment, the staggered fashion may be dictated by the stage or phase of the disease.

In one embodiment, the micro-organ is implanted at a desired location in the subject in such a way that at least a portion of the cells of the micro-organ remain viable. In one embodiment of this invention, at least about 5%, in another embodiment of this invention, at least about 10%, in another embodiment of this invention, at least about 20%, in another embodiment of this invention, at least about 30%, in another embodiment of this invention, at least about 40%, and in another embodiment of this invention, at least about 50% or more of the cells remain viable after administration to a subject. The period of viability of the cells after administration to a subject can be as short as a few hours, e.g., twenty-four hours, to a few days, to as long as a few weeks to months or years.

Micro-organ implantation within a recipient subject provides for a sustained dosage of the recombinant product. The micro-organs may be prepared, prior to implantation, for efficient incorporation within the host facilitating, for example, formation of blood vessels within the implanted tissue. Recombinant products may therefore be delivered immediately to peripheral recipient circulation, following production. Alternatively, micro-organs may be prepared, prior to implantation, to prevent cell adherence and efficient incorporation within the host. Examples of methods that prevent blood vessel formation include encasement of the micro-organs within commercially available cell-impermeant diameter restricted biological mesh bags made of silk or nylon, or others such as, for example GORE-TEX bags (Terrill P J, Kedwards S M, and Lawrence J C. (1991) The use of GORE-TEX bags for hand burns. Burns 17(2): 161-5), or other porous membranes that are coated with a material that prevents cellular adhesion, for example Teflon.

Gene products produced by micro-organs can then be delivered via, for example, polymeric devices designed for the controlled delivery compounds, e.g., drugs, including proteinaceous biopharmaceuticals. A variety of biocompatible polymers (including hydrogels), including both biodegradable and non-degradable polymers, can be used to form an implant for the sustained release of a gene product of the micro-organs in context of the invention at a particular target site. The generation of such implants is generally known in the art (see, for example, Concise Encyclopedia of Medical & Dental Materials, ed. By David Williams (MIT Press: Cambridge, Mass., 1990); Sabel et al. U.S. Pat. No. 4,883,666; Aebischer et al. U.S. Pat. No. 4,892,538; Aebischer et al. U.S. Pat. No. 5,106,627; Lim U.S. Pat. No. 4,391,909; and Sefton U.S. Pat. No. 4,353,888).

Implantation of genetically modified micro-organs according to the present invention can be effected via standard surgical techniques or via injecting micro-organ preparations into the intended tissue regions of the mammal utilizing specially adapted syringes employing a needle of a gauge suitable for the administration of micro-organs. In another embodiment, a catheter is employed for implanted micro-organs. In one embodiment, any of the implantation methods described in PCT Publication WO2 04/099363 may be used and is considered an embodiment of this invention.

In one embodiment, micro-organs are implanted subcutaneously, intradermally, intramuscularly, intraperitoneally or intragastrically. In one embodiment, the term implanted excludes being grafted as a split-thickness or full-thickness skin graft. In one embodiment of the present invention, the donor micro-organs utilized for implantation are preferably prepared from an organ tissue of the recipient mammal (i.e. autologous). In one embodiment, a Biopump is not encapsulated or inserted into a biodepgradable device.

In one embodiment, while implantation of a GM-DMO-IFN of the present invention may result in a sustained nucleic acid expression, polypeptide secretion, increased half-life of a polypeptide and/or sustained biological response, a sustained therapeutic response may not correlated directly to a sustained, elevation of the expressed protein.

Without being limited by theory, in one embodiment, levels of nucleic acid or polypeptide expressed by the long-lasting formulations of the present invention may decrease as a function of time as a result of the death of differentiated dermal fibroblasts expressing the recombinant nucleic acid or polypeptide.

In contrast to other methods involving transient transduction of cells, or cells that turn over rapidly, the GM-DMO-IFN of the instant invention comprises cells that are infrequently replicated. Therefore, the GM-DMO-IFN produces a stable protein from a stable construct and is expected to continue producing the protein already characterized for a sustained period of time.

It may be that the response to implantation of an at least one GM-DMO-IFN does not sustain or maintain the biological response, e.g. reduction of viral load. In such a case, additional GM-DMO-IFN may be implanted in the subject.

In one embodiment, a method of this invention comprises a step of implanting at a later date to said subject, an additional at least one genetically modified autologous micro-organ that expresses and secretes IFN. In one embodiment, the at least one genetically modified autologous micro-organ is a dermal micro-organ. In one embodiment, the genetically modified autologous micro-organ comprises a helper-dependent adenovirus vector comprising a nucleic acid sequence encoding at least the IFN therapeutic polypeptide or functional portion thereof. In another embodiment, the genetically modified autologous micro-organ comprises an adeno-associated viral vector comprising a nucleic acid sequence encoding at least the IFN therapeutic polypeptide or functional portion thereof.

Treatment by implanting GM-DMO-IFN, aims to supply a steady continuous production and delivery of IFN or a functional portion thereof, to patients in need. Implantation of autologous GM-DMO-IFN back into a patient, wherein the autologous tissue remains localized and supplies sustained treatment, may provide this benefit. A strong advantage of this method is that if the delivered dose of IFN is too high, or if the treatment needs to be terminated for any reason, one or more of the implanted GM-DMO-IFN(s) may be simply removed (or even potentially ablated in situ) in order to stop the production and delivery of the IFN.

### EXAMPLES

### Experimental Materials and Methods

### Materials and Equipment List

Production medium was used to grow micro-organs and comprises DMEM-HEPES Medium (High glucose 4,500 mg/L and 25 mM HEPES; Hi-Clone Cat# SH3A1448.02) comprising 1% glutamine and supplemented with 50 μg/ml Gentamycin (RAFA labs, for injection) and 0.1% Amphotericin B (BMS, Fungizone I.V.) (final concentration in the media 2.5 μg/ml Amphotericin B). In some experiments, 10% serum substitute supplement (SSS, Irvine Scientific, Cat #99193), 10% autologous human serum, or 10% Fetal bovine serum (FBS or FCS) was added to the production medium.

### Harvesting of Dermal Micro-Organs

A. Method I.

Human dermal micro-organs were harvested from an area of skin from a region of the donor's lower abdomen. In certain instances, the dimensions of the dermal micro-organs harvested were approximately 1.5-2.5 mm in diameter and 30 mm in length. In some cases, to prevent the harvest of the epidermis, a shallow slit (1-2 mm deep) passing through the stratum cornea into the dermis was cut along a straight line at one side of the skin region from which the micro-organs were to be harvested, and a similar slit was cut 30 mm away from and parallel to the first slit. The distance between the slits determined the micro-organ length and was consistent throughout the experiments.

In certain cases, a thin gauge (typically 22GA) hypodermic needle attached to a 1 ml syringe filled with sterile saline was inserted into the exposed dermis at the first slit and slid along the dermis of the harvesting site towards the opposite slit, with the needles angled as necessary so that it exited through the dermis at the opposite slit.

Next, in some cases, the outer skin along the length of the guiding needle was pinched with a surgical clamp. The needle embedded in the dermis was lifted slightly to raise the area of skin surrounding it and sometimes a hook shaped device beneath the inserted hypodermic needle's point was used to assist in lifting the skin before it's pinched. The tip of the guide needle protruding from its point of exit, was inserted into the sharp leading end of a coring needle (1-3 mm in diameter, Point Technologies, CO USA), which was held by a commercially available drill (such as Aesculap Micro Speed GD 650, GD 657). A small amount of sterile saline was injected from the syringe into the coring needle. The drill is activated to rotate the coring needle at high speed (typically 3000-7000 RPM) and while rotating, the drill and coring needle are manually urged forward along the axis of the guide needle to cut a 30-40 mm long cylindrical dermal core (dermal micro-organ) having an outer diameter approximately that of the inner diameter of the coring needle. The dermal micro-organ usually remained attached to the guide needle, which was withdrawn from within the coring needle and placed in Production media (as described hereinabove), and the coring needle was removed from the skin.

In many cases, using tweezers, each micro-organ was transferred to a labeled single well in a 24 well plate containing 1000 μl Production Medium. To remove the debris, two additional media changes of 1000 μl were performed for each micro-organ. The plates containing the micro-organs in 1000 μl production media were then transferred to an incubator that had been equilibrated to 32° C., 10% CO₂, and ˜95% humidity for a 24 hr recovery period.

A. Method II.

Harvesting methods of dermal micro-organs using a sterile support structure and associated components are described in detail in U.S. application Ser. No. 13/686,939, which is incorporated herein in full.

Briefly, with the subject prone, a harvest site on the lower abdomen was selected, disinfected, marked with guidelines, and injected with local anesthesia. The harvest site was in an area of healthy skin, free of stretch marks or other obvious skin abnormalities. A sterile harvesting support structure containing a vacuum control hole was connected to a vacuum source, the vacuum turned on and the support structure placed on the subject's epidermis at the selected harvest site with the vacuum control hole uncovered.

A finger was placed over the vacuum control hole causing a vacuum to raise the skin-related tissue structure into the vacuum chamber.

An introducer needle was used to puncture the skin at the harvest site and insert a sleeve through which a coring needle could later be introduced into the supported skin tissue. The introducer needle was then removed, leaving the sleeve in place.

Next the symmetrically sharpened tip of the coring needle attached to a medical drill was inserted through the sleeve and gently pushed forward until the tip reached the distal end of the sleeve. The drill was then activated and pushed forward to the full stop, pushing the tip of the coring needle through the dermal tissue and into fat tissue. At this point the vacuum was deactivated by removing the finger from the vacuum control hole.

The drill was then disconnected from the coring needle and the DMO sterilely collected.

B. Methods Following Harvest

In some embodiments, micro-organs (MOs) were harvested under local anesthesia from the dermis of the lower abdomen or the upper or lower back of the patient that will be treated, with dimensions of approximately 1.5-2.5 mm in diameter, and 30 mm in length. Their dimensions and appearance remain essentially the same during the entire hEPO-GMMO or hIFN-GMMO Biopump ex-vivo production process.

The dermis micro-organs were transported under controlled conditions to a contract cGMP (current Good Manufacturing Practice) processing facility. Upon receipt at the cGMP processing center, the MOs were divided into two subsets: those to be processed immediately into Biopumps, and those that will be cryopreserved for later processing, if required, into Biopumps. The decision as to how many will be processed immediately was based on an estimate of the EPO or IFN dose needed for a particular subject and an estimate of average secretion levels from typical Biopumps based on thousands of processed Biopumps in pre-clinical and clinical testing.

For the MOs that were processed immediately, the HDAd-EPO vector or HDAd-IFN vector was used to perform the transduction. In some instances, MOs were transduced after 24 hours of harvest. In other instances, MOs were transduced were transduced after 72 hours from harvesting. After transduction, residual viral particles were removed by several media exchanges. The Biopumps (transduced micro-organs) were maintained in culture for approximately one week in order to assay for protein secretion levels and sterility.

The Biopumps were then transported under controlled conditions back to the treatment center for subcutaneous implantation under local anesthesia in the abdominal wall or the upper or lower back of the same patient or for implantation in SCID mice.

Micro-organs retrieved from cryostorage for processing, if required, into Biopumps are first thawed prior to undergoing the transduction and maintenance procedures, as detailed above. Data demonstrate the comparability of Biopumps produced from fresh and cryopreserved MOs.

The decision as to how many dermal micro-organs are transduced at any given time, is based upon the estimated dose needed for the patient/SCID mouse and an estimate of average secretion levels from typical genetically modified dermal micro-organs. Dermal micro-organs not used immediately for transduction are cryopreserved for later processing

C. Cell Viability Following Harvest.

Viable counts of cells from collagenase dispersed hIFN-Biopumps on day 1 and day 9 post-harvest were made (FIG. 10). MOs were harvested from the excised skins of six sequential abdominoplasty subjects. One day later and nine days later, four to five MOs were dissociated using collagenase, and viable cells were manually counted three times from three independent samples by trypan blue exclusion using a cell counter chamber.

FIG. 10 shows no significant change in the number of viable cells in a given MO between day 1 and day 9 post harvesting, indicating that MOs maintain their viability during the time period between harvesting and implantation. Sustained cell viability during the first nine days post-transduction was confirmed using a Glucose assay kit (Sigma GAGO-20) to measure glucose consumption per hIFN-Biopump (data not shown).

### Virus Transduction

Each micro-organ was transferred for transduction into a well of a 48-well plate, which has smaller wells requiring smaller total fluid volume, to conserve virus solution. The medium was carefully removed from each well without disturbing the micro-organ inside. During preclinical experiments, three different vectors were tested: 1.6-3×10⁹ infectious particles (ip)/ml of first generation adenovirus (Molecular Medicine), approximately 3-4×10¹² viral particles/ml helper-dependent adenovirus (Baylor), or approximately 2×10¹¹ viral particles/ml adeno-associated virus (University of Pennsylvania), each comprising recombinant human EPO gene, optimized recombinant human EPO gene, or optimized IFN-alpha gene, were each diluted 1:10, 1:25, 1:50, 1:100, 1:500, or 1:1000 in DMEM-HEPES (Gibco Cat#42430-025) with or without FCS. Each well of the 48-well plates was filled with 100 μL of one of the diluted titers of a virus. The plate was placed in a CO₂ incubator and transduction was assisted by agitation on a digital microtiter shaker at 300 rpm for a period of 2 hours and an additional 16-22 hour incubation without shaking.

The transduced micro-organs (Biopumps) were transferred to a 24-well plate after transduction and then washed three times with 1 mL production media (without FCS) to remove the non-transduced viral particles. After washing, the biopumps were maintained in 1 mL production media in a standard high humidity CO₂ incubator at 95% humidity, 10% CO₂, and 32° C. Seventy-two hours after the removal of the viral vector, the production medium was replaced with fresh medium, and aliquots of the spent medium were assayed for secreted recombinant protein levels.

The delivery vector for the Examples below, used to transduce the cells of MOs biopsied from dermis, is a non-replicating helper-dependent Adenoviral (HDAd) vector.

In the case of a Biopump expressing interferon (hIFN-Biopump), the expression cassette within the HDAd vector includes, but is not limited to, a nucleotide sequence encoding interferon, a CAG promoter and a SV40 poly A sequence. The nucleic acid sequence encoding interferon includes, in some instances, SEQ ID No. 1. In other instances, the nucleic acid sequence encoding interferon includes, in some instances, SEQ ID No. 4. In certain cases, the nucleic acid sequence of the transduction vector, including the nucleic acid sequence encoding interferon, a CAG promoter and a SV40 poly A sequences, includes SEQ ID No. 2.

FIG. 9 shows a clear correlation between the viral titer used in transduction and the resulting in-vitro IFNα secretion levels from hIFN-Biopumps, confirming that hIFN-Biopumps of different potency can be produced. MOs were produced from skin of a single donor, and transduced with HDAd-IFNα titers corresponding to 3×10¹⁰ (leftmost bar in each grouping), 1.5×10¹⁰ (second to leftmost bar in each grouping), 5×10⁹ (second to rightmost bar in each grouping), and 1×10⁹ (rightmost bar in each grouping) vp/MO. Free viral particles were then washed away and the hIFN-Biopumps were maintained per standard protocol. Media was collected and IFNα levels were tested by ELISA.

In clinical use, a single titer would be used to transduce all MOs, and the resulting daily IFNα production would be measured for each Biopump. The total administered dose of hIFN-Biopumps producing sustained secretion of IFNα would then be adjusted by implanting the requisite number of Biopumps of measured potency.

### Ex Vivo Micro-Organ Maintenance

Every 3-4 days, used production media was collected, and the level of the secreted recombinant protein and glucose level were assessed along with the viability of the Biopumps. Fresh Production media was added to the 24-well plate.

Table 2 shows that there was no significant change in the number of viable cells pre- and post-transduction, indicating that neither viral transduction nor secretion of EPO or IFNα impacts the viability of an EPO- or IFN-Biopump, respectively.

TABLE 2

| | Days from | |
|---|---|---|
| Treatment | Harvest | % Viable cells |
| No Virus | 0 | 90.8 ± 1.5 |
| No Virus | 35 | 93.3 ± 1.1 |
| EPODURE Biopump | 35 | 91.4 ± 1.4 |
| INFRADURE Biopump | 35 | 86.2 ± 4.8 |

Table 2 shows the percent (%) viable cells of micro-organs and Biopumps on days 0 and 35 post-harvesting. On days 0 (non-transduced MO only) and 35 post-harvesting, samples were dissociated using collagenase and viable cells were manually counted by trypan blue exclusion three times from three independent samples using a cell counter chamber.

### Secreted Protein Measurements

Human EPO (hEPO) and IFNα concentration and secretion levels were assayed using an enzyme-linked immunosorbent assay (ELISA) kit (Quantikine human erythropoietin; R&D Systems; Human interferon alpha ELISA kit, PBL Biomedical Laboratories), according to the manufacturer's instructions.

### Glucose Measurements

Tissue glucose consumption was evaluated using Sigma-Aldrich Corporation GAGO20 Glucose (GO) Assay Kit, according to manufacturer's instructions.

### Hematocrit Measurements

Hematocrit levels were assayed using centrifugation according to the reference method recommended by The National Committee for Clinical Laboratory Standards (NCCLS), as is known in the art. To determine the hematocrit, whole blood in a tube was centrifuged at 10-15,000×g for 5 minutes to pellet the red cells (called packed erythrocytes), and the ratio of the column of packed erythrocytes to the total length of the sample in the capillary tube was measured with a graphic reading device within 10 minutes of centrifugation.

### Micro-Organ Implantation

In some experiments, genetically modified or non-transduced control micro-organs were implanted subcutaneously in Severe Combined ImmunoDeficiency (SCID) mice after assaying tissue glucose consumption to ascertain that micro-organs were viable. Male and female SCID mice weighing around 25 grams were anaesthetized with 140 μl of diluted Ketaset (ketamine HCl) (400 μl Ketaset and 600 μl saline) and control or EPO-expressing or IFN-expressing micro-organs were implanted subcutaneously ten days following micro-organ transduction.

### Example 1

### EPO and IFNα Levels Produced In Vitro by GMMOs

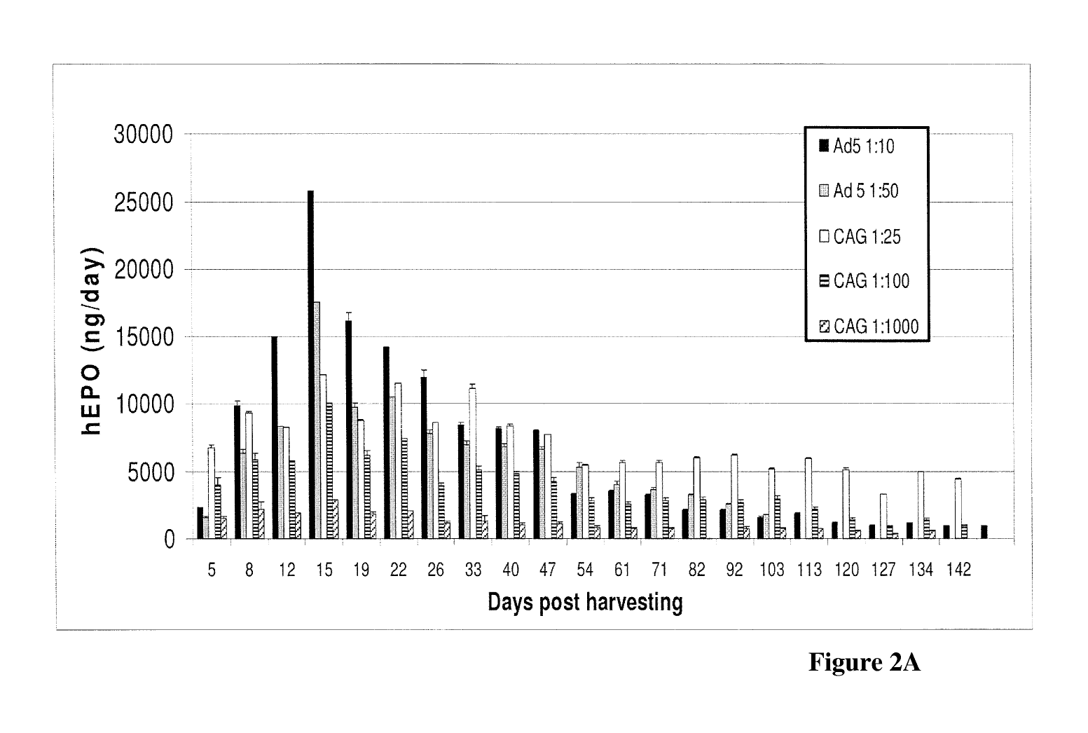

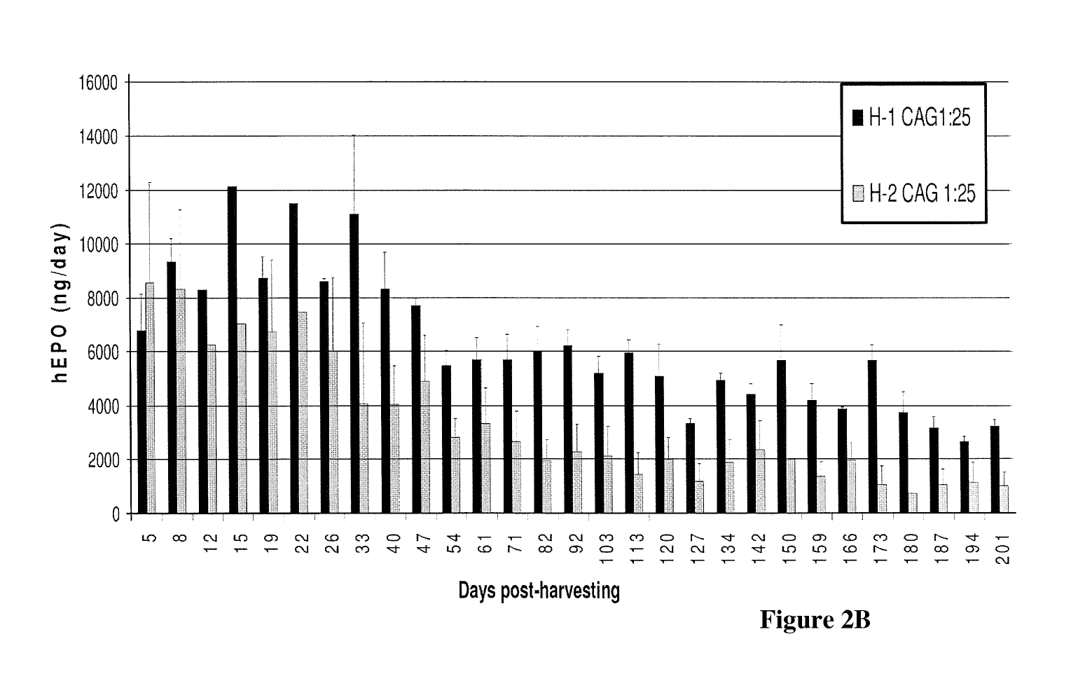

Micro-organs were prepared as described above and transduced with a helper-dependent adenoviral vector expressing an optimized IFNα gene linked to a CAG promoter, as described above. GMMOs were then maintained in culture, and the levels of IFNα produced were evaluated by ELISA. Optimized IFNα-expressing micro-organs produced greater than 1000 ng/day of IFNα in vitro (FIG. 1) for at least 40 days post-harvesting, and recombinant hEPO-expressing micro-organs produced greater than 1000 ng/day of hEPO in vitro (FIGS. 2A-B) for at least 142 days post-harvesting.

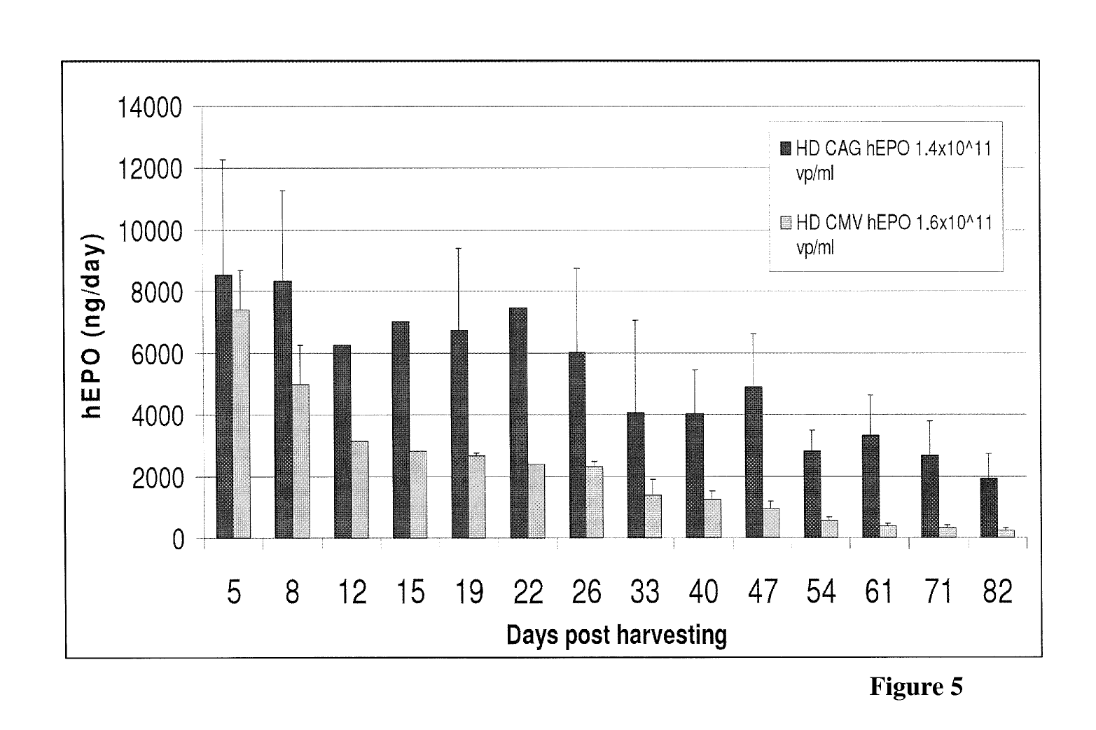

GMMOs comprising a gutless adenovirus vector encoding optimized hEPO maintained higher percentages of peak expression for more than 200 days compared to micro-organs comprising an adenovirus-5 vector encoding hEPO (FIG. 3). Micro-organs comprising a gutless adenovirus vector encoding optimized hEPO also maintained a higher percentage of peak expression for a longer period of time than micro-organs comprising a gutless adenovirus vector encoding non-optimized hEPO (FIG. 4). Finally, micro-organs comprising a gutless adenovirus vector encoding hEPO downstream of a CAG promoter showed higher levels of hEPO expression, which grew more pronounced as a function of post-transduction day, compared to micro-organs comprising a gutless adenovirus vector encoding hEPO downstream of a CMV promoter (FIG. 5).

GMMOs comprising a gutless adenovirus vector encoding optimized hIFN showed sustained IFNα production when maintained under standard cell culture conditions (FIG. 7). FIG. 7 shows that an hIFN-GMMO produced on average 2-3 μg hIFN/day for greater than six months. This level of hIFN secretion is equivalent to approximately 0.7-1 million IU/Biopump and 9 million IU/week.

Daily in vitro production of hIFN on day 10 post harvesting, from hIFN-Biopumps from seven subjects is shown in FIG. 8. FIG. 8 shows hIFNα secretion levels of 2,500-5,000 ng/Biopump/day. The standard deviation of expression from each Biopump was quite moderate, indicating that the variability of secretion levels from hIFN-Biopumps of the same skin sample was small.

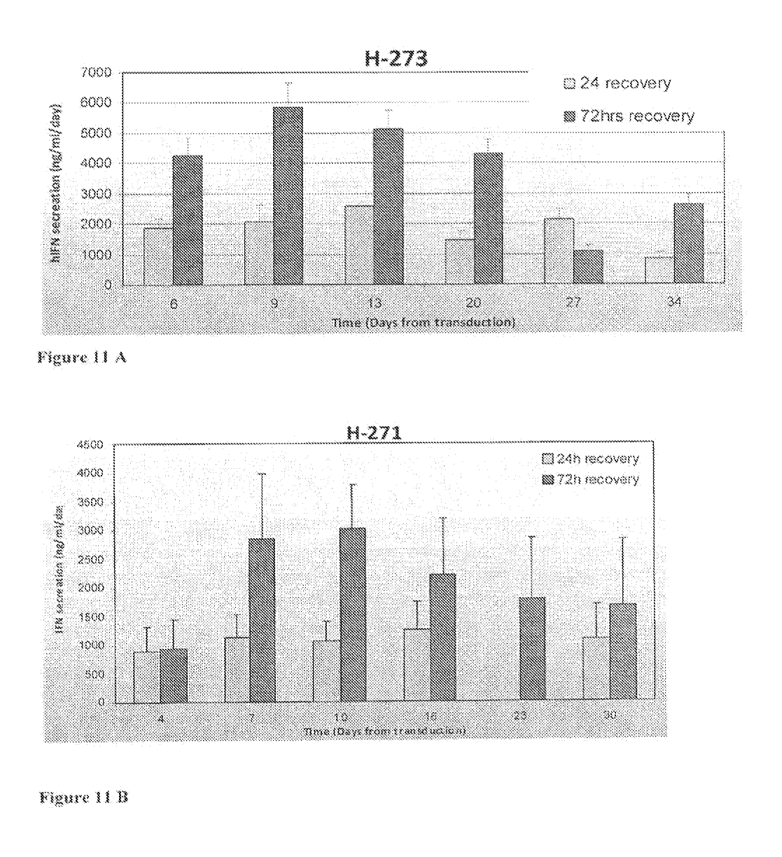

Unexpectedly, the extension of the latency period between harvest and transduction proved beneficial for IFNα production/secretion. FIGS. 11A and 11B demonstrate in hIFN-Biopumps from two different skin samples that transductions following a 72 hour recovery period following harvest resulted in significantly increased IFNα secretion compared to transduction after a 24 hour recovery following harvest.

### Example 2

### EPO Levels Produced by Human EPO-Expressing GMMOs Maintained In Vitro and in Serum of Implanted SCID Mice

EPO-expressing micro-organs were prepared as described above. After a total of nine days in culture, the amount of EPO produced per micro-organ was measured, and this value was used to determine that each mouse was implanted with micro-organs expressing equivalent levels of EPO. On the tenth day, two micro-organs were implanted subcutaneously into each SCID mouse and on the first measurement taken after ten days, levels of hEPO measured in the serum of the SCID mice were significantly above baseline levels. The levels remained high at least 216 days post-implantation and significantly raised hematocrit levels in SCID mice for at least 157 days (FIG. 6A). Non-implanted EPO-expressing micro-organs produced from the same donor at the same time as the implanted EPO-expressing micro-organs but maintained in vitro continuously maintained high levels of EPO production (FIG. 6B). Micro-organs transduced with vectors comprising optimized hEPO gene produced higher levels of EPO than those transduced with recombinant hEPO gene both in vivo (FIG. 6A) and in vitro (FIG. 6B). Control SCID mice implanted with non-EPO-producing micro-organs showed no increase of serum EPO levels and no significant changes in hematocrit levels after micro-organ implantation compared to pre-implantation (FIG. 6A). Micro-organs comprising EPO-expressing adenovirus-5, which was used as a positive control, was used at a titer of 1:10 compared to a titer of 1:100 for micro-organs comprising EPO-expressing optimized or non-optimized gutless adenovirus.

### Example 3

### IFN Levels Produced by Human IFNα-Expressing GM-MOs in Serum of Implanted SCID Mice

IFNα-expressing micro-organs were prepared as described above. Dose dependent delivery of hIFN-Biopumps in SCID mice was analyzed. Briefly, MOs were harvested from the dermis of excised skin from healthy individuals undergoing routine abdominal resection (tummy-tuck operations), and were then transduced with HDAd-IFNα (hIFN-Biopump). Following transduction, residual virus was washed away and hIFN-Biopumps were maintained in culture media in a CO₂ incubator. Culture media was replaced every three to four days. The hIFN-Biopumps were subcutaneously implantated in SCID mice to deliver either a low dose of 1,300 ng/day to each of four mice, or a high dose of 4,000 ng/day to each of two mice. Blood samples were taken on the day of implantation, and thereafter every 10-14 days for a total of 105 days.

FIG. 12 documents the in vivo dose response to hIFN-Biopump by SCID mice. Serum IFNα initially reached levels appropriate for 70 kg humans rather than 20 g mice; on day 10: the low dose group averaged 1700 pg/mL, and the high dose group averaged 8,900 pg/mL. Secretion of IFNα from the single administration of hIFN-Biopump, and a difference in serum IFNα levels between the low and high dose groups, was maintained throughout the duration of the experiment (over 100 days). Although a steady decline in serum concentration is seen over several weeks, the levels were maintained above standard therapeutic levels of 10 pg/mL for over three to four months.

FIGS. 13 and 14 document consistent substantial levels of active IFNα in the serum of SCID mice ten days post-implantation with hIFN-Biopumps, indicating in vivo delivery of potent IFNα by hIFN-Biopump. Briefly, twelve SCID mice were implanted with hIFN-Biopumps secreting at least 1 μg/day of human IFNα. Blood samples were taken prior to implantation and ten days post-implantation.

In FIG. 13 the bars correspond to serum IFNα levels measured by ELISA (pg/ml), and diamonds correspond to serum IFNα bio-activity measured by bioluminescence of IFNα bioactivity in interferon-sensitive cells (iLite Human Interferon Alpha kit, PBL InterferonSource Product #51100).

FIG. 14 shows the correlation between serum IFNα by ELISA versus serum IFNα bioactivity on the same samples shown in FIG. 13. Each point represents a measurement from a single mouse. Pre-implantation serum IFNα levels and activities were 0, and on day 10 serum IFNα ranged from about 3,000 to 6,000 pg/ml, correlating well to activity ranging from about 1,300 to 2,500, with R2=0.965. Y/X ratio was 2.28 pg/IU, indicating potency compares favorably to the 3.85 pg/IU of commercially available IFNα (Intron-A, per its package insert).

### Example 4

### Dose Escalation Study of the Safety, Tolerability and Efficacy of the IFN-Biopump Secreting Sustained Interferon Alpha-2b (IFNα)

Materials: autologous dermal Biopumps producing and secreting IFNα-2b.

Subjects: newly diagnosed with hepatitis C virus (HCV) genotype 2 or 3 infection liver disease. Subject age range is 18-60.

Methods: Subjects will have implanted autologous dermal Biopumps producing and secreting escalating doses of IFNα-2b, as a component of HCV treatment Implantation will be subcutaneous or intradermal implantation. Treatment may be in combination with oral ribavirin. Treatment will be over a period of up to 24 weeks with an additional 24 weeks of safety follow-up.

Results: Assessment of safety and tolerability will be measured by adverse events (AEs), laboratory data, physical examinations, vital signs, wound healing assessments, and localized skin inflammation (using Draize score).

Assessment of efficacy will be measured based on the sustained elevation of serum IFN, virologic response and change in biomarkers (Beta 2 Microglobulin, 2.5 OAS and Neopterin).

Assessment of dose response will compare between serum IFNα elevation following administration (implantation) of the autologous Biopump expressing and secreting IFNα and the administered dose of autologous Biopump expressing and secreting IFNα dose as measured in vitro prior to implantation.

The time-line for this study is presented below in Table 3.

TABLE 3

| Period No. | Period Description | Timing/Duration |
|---|---|---|
| I | Screening | 6 weeks to 11 (+1 to 4) days |
| | | prior to Day 0 |
| II | Harvest and Biopump production | 12 to 16 days prior to |
| | | Implantation Visit (Day 0) |
| III | Treatment Initiation | Day 0 to end of Treatment |
| | GM-DMO-IFN Biopumps | Week (TW) 24 |
| | implantation | |
| | Initiation of oral Ribavirin | |
| | (Copegus) treatment | |
| | Initiation of viral response | |
| | assessment. | |
| IV | End of treatment and Safety | Week 25 to 48 |
| | Follow-up | |
| | Safety follow-up from treatment | |
| | termination to end-of-study | |
| | (EOS) | |

Screening: The screening visit (Visit 1) will include informing the patient of all details of the study, allowing to ask all relevant questions and read the informed consent form (ICF) and through signature. Then blood will be drawn for laboratory tests, medical history will be collected and documented, a complete physical examination will be performed including ECG, and the documentation of concomitant medications. The most appropriate anatomical site for harvesting will be initially selected during the physical examination and confirmed by the surgeon prior to the procedure.

The final decision regarding enrollment will be performed at the clinical site after review of the lab results.

Once found eligible for the study, subjects will enter the IFN-Biopump production period in which subjects will be forwarded for harvesting of MOs at the clinical site.

The entire IFN-Biopump production process, from harvest to implantation, takes 14 days±2 days.

At Visit 2, the Harvesting visit, just prior to the harvesting procedure, blood samples will be withdrawn for lab tests of hematology, serum IFNα and quantitative HCV RNA. Medical history and concomitant medications will be evaluated for any changes since screening. Local anesthesia will be applied at the target harvest site. 10 dermal core tissue samples, approximately 30 mm long, will be removed using a harvesting device with a 14 gauge coring needle in an ambulatory procedure room at the clinical center. The harvesting device will use a commercially available medical drill. The harvest site will be evaluated, bandaged, and the subject will be monitored for up to two (2) hours following harvest. The subject will be discharged with written instructions on wound and pain management.

The dermal samples will be transferred in a sterile manner to the GMP production facility.

One of the harvested dermal cores will not be transduced with the HDAd viral vector, but will be otherwise processed as the other Biopumps, to provide a non-transduced control. IFN-Biopump quality will be assayed by performing sterility tests and ELISA assays to determine the average daily IFNα secretion per IFN-Biopump. IFNα production by each IFN-Biopump prior to implantation is used as its potency to determine the number of IFN-Biopumps to implant in the subject to reach the desired total dose per day.

The IFN-Biopump implantation procedure will be performed at visit 3 (day 0) in an ambulatory procedure room at the clinical center. Medical history and concomitant medications will be evaluated for any changes since screening. Local anesthesia will be applied at the target implantation sites. Implantation of the desired number of IFN-Biopumps, determined based on the target dose and the potency of each individual IFN-Biopump, will be performed by intradermal or subcutaneous injection using a standard trocar procedure together with an implantation tool based on a variation of the harvesting tool to assist in controlling the depth of implantation. The non-transduced dermal core will also be implanted at the implantation visit.

The implantation/harvest sites will be marked, evaluated and bandaged, and the subject will be monitored for up to two (2) hours following implantation. The healing process of the harvest and implantation sites will be observed and findings documented weekly until week four. Oral ribavirin will be initiated the day following implantation.

Following implantation subjects will visit the clinical site at days 1, 3, 5, 7, 9, 11, and 14, and then once every two weeks until week 12. Thereafter, subjects will visit the clinical site once every four weeks until end of treatment and throughout the safety follow-up period. All visits will have the flexibility to be performed ±3 days of the scheduled date.

Blood samples will be drawn for serum IFNα2b and HCV RNA viral load at each clinic visit. Biochemistry including liver function panel and hematology will be evaluated monthly until end of treatment period.

During the treatment period, patients will receive dual therapy of IFN-Biopump and oral Ribavirin.

Stopping Rules:

(1) If at any time as of treatment day 14, the HCV RNA viral load increases by ≧1 Log from the nadir HCV RNA, and is confirmed in two consecutive tests 2 weeks apart, the subject's participation in the study will be terminated, and alternative treatment will be administered as per the decision of the treating physician. Nadir HCV RNA is defined as the lowest value seen during the period from Baseline throughout the first 14 days post Biopumps implantation. (2) If HCV-RNA is detectable at TW 12, the subject's participation in the study will be terminated, and alternative treatment will be administered as per the decision of the treating physician. (3) If HCV-RNA is detectable after TW12 at two consecutive tests, up to 1 month apart, the subject's participation in the study will be terminated and alternative treatment will be administered as per the decision of the treating physician.

The excision of the IFN-Biopumps at end of treatment is not mandatory. If IFNα levels are at baseline, it is assumed that there is no IFNα production/secretion and the autologous Biopumps will not be excised at the termination of dual therapy.

If the Biopumps must be excised, the excision procedure will take place during TW 25. The marked implantation sites will be excised by a surgeon and the Biopumps will be sent for histology and immunohistochemistry for IFNα (if feasible) evaluation. The excision sites will be evaluated based on the Draize score and for hematomas, bandaged, and the subject will be monitored for up to two (2) hours following excision, during which time vital signs will be measured every 60 minutes. As per surgeon's advice, the patient will visit the site for suture removal if required and for evaluation of the excision site.

After end of treatment, visits will take place every 4 weeks until week 48.

Dosing:

There will be three dosage groups of four subjects each. Group 1 receives 3×10⁶ IU/day. Group 2 receives 5×10⁶ IU/day. Group 3 receives 9×10⁶ IU/day.

The in vitro production of IFNα by each Biopump on the day of product release serves as a basis for target dose for implantation. The aggregate number of IFNα units implanted will provide ±25% of the designated target dose as stated above.

Dosing with Oral Ribavirin: All subjects will be treated concomitantly with oral ribavirin 800-1200 mg daily. Dose will be based on genotype and weight as per standard of care (SOC).

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A long-lasting therapeutic interferon formulation comprising at least one genetically modified micro-organ that expresses and secretes interferon, said genetically modified micro-organ comprising a vector comprising a nucleic acid sequence encoding an interferon operably linked to one or more regulatory sequences, wherein said nucleic acid encoding interferon comprises SEQ. ID. No. 1.

2. The formulation of claim 1, wherein said at least one genetically modified micro-organ is a genetically modified dermal micro-organ.

3. The formulation of claim 1, wherein said regulatory sequence comprises a CAG promoter.

4. The formulation of claim 1, wherein said regulatory sequence comprises a CMV promoter.

5. The formulation of claim 1, wherein said regulatory sequence comprises a SV40 polyadenylation sequence.

6. The formulation of claim 1, wherein said micro-organ is transduced with a viral vector selected from the group consisting of an adeno-associated viral (AAV) vector and a helper dependent adenoviral (HDAd) vector.

7. The formulation of claim 6, wherein said helper-dependent adenoviral vector comprises SEQ ID. No. 2.

8. The formulation of claim 6, wherein said transduction is performed at least 24 hours after harvesting said micro-organ.

9. A method of treating hepatitis in a human subject in need over a sustained period of time comprising the steps of:
a. providing at least one genetically modified micro-organ that expresses and secretes interferon, said genetically modified micro-organ comprising a nucleic acid sequence encoding interferon operably linked to one or more regulatory sequences;
b. determining interferon secretion levels of said at least on genetically modified micro-organ in vitro;
c. implanting said at least one genetically modified micro-organ in said human subject at an effective dosage; and either
d. measuring interferon in the serum of said human subject, or
e. measuring levels of hepatic virus in said human subject.

10. The method of claim 9, wherein said interferon is selected from the group including interferon alpha, interferon beta and interferon gamma.

11. The method of claim 10, wherein said interferon alpha is interferon alpha 2a or interferon alpha 2b.

12. The method of claim 9, wherein said nucleic acid sequence encoding said interferon is optimized for increased expression levels, increased duration of expression, or a combination thereof.

13. The method of claim 12, wherein said optimized nucleic acid sequence is greater than 85% homologous to SEQ ID No: 1.

14. The method of claim 9, wherein said micro-organ is transduced with a viral vector selected from the group consisting of an adeno-associated viral (AAV) vector and a helper dependent adenoviral (HDAd) vector.

15. The method of claim 14, wherein said helper-dependent adenoviral vector comprises SEQ ID. No. 2.

16. The method of claim 14, wherein said transduction is performed at least 24 hours after harvesting said micro-organ.

17. The method of claim 9, wherein said genetically modified micro-organ is a genetically modified dermal micro-organ.

18. The method of claim 9, wherein said hepatitis is hepatitis B.

19. The method of claim 9, wherein said hepatitis is hepatitis C.

20. The method of claim 19, wherein said hepatitis C is genotype 1 or genotype 2 or genotype 3, or any combination thereof.

21. The method of claim 9, wherein said hepatitis is hepatitis D.

22. The method of claim 21, wherein said hepatitis D is chronic hepatitis D.
